(19) United States
(12) Patent Application Publication
(10) Pub. No.: US 2011/0166417 A1
(43) Pub. Date: Jul. 7, 2011

(54) METHOD AND DEVICE FOR REPAIR OF DEGENERATIVE CARTILAGE

(75) Inventor: Chia-Ying Lin, Ann Arbor, MI (US)

(73) Assignee: THE REGENTS OF UNIVERSITY OF MICHIGAN, Ann Arbor, MI (US)

(21) Appl. No.: 12/863,265

(22) PCT Filed: Jan. 21, 2009
(86) PCT No.: PCT/US2009/031545

§ 371 Date: Mar. 21, 2011

Related U.S. Application Data
(60) Provisional application No. 61/022,061, filed on Jan. 18, 2008.

(51) Int. Cl. A61B 1/06; A61K 38/18; A61K 9/00

(52) U.S. Cl. 600/104; 514/8.8; 424/400

(57) ABSTRACT

The present invention relates, in general to methods for treating degenerative cartilage in an individual comprising administering a composition that increases bone morphogenic protein (BMP) expression directly into injured or damaged cartilage, such as in a vertebral disc or a joint, wherein the composition is in a controlled release formulation.

This application claims the priority benefit of U.S. Provisional Application No. 61/022,061, filed, Jan. 18, 2008, herein incorporated by reference.

### FIELD OF THE INVENTION

The present invention relates, in general, to methods to repair spinal disc and other cartilage damage comprising administering a compound that increases bone morphogenic protein expression in a controlled release formulation directly to the damaged cartilage, i.e., in a vertebral disc or joint. Also contemplated are compositions useful in the method.

### BACKGROUND OF THE INVENTION

Intervertebral disc (IVD) degeneration refers to a complex process involving altered tissue composition induced by aberrant cell-mediated responses that eventually lead to progressive structural failure of spinal balance (1). The disc cells residing in both nucleus pulposus (NP) and annulus fibrosus (AF) actively regulate the homeostasis of disc tissue by maintaining equilibrium between the synthesis and breakdown of the extracellular matrix, which mainly consists of large aggregates of proteoglycan and collagen (2). Nucleus pulposus consists of chondrocytes, collagen fibrils, and proteoglycan aggrecans (U.S. Patent Publ. 20070003525), while annulus fibrosus comprises concentric sheets of collagen fibers, connected to the vertebral end plates, and encloses the nucleus pulposus. Disc degeneration often starts with cellular and biochemical changes, mostly due to aging or injury, resulting in an imbalance between anabolism and catabolism of disc tissues. As a consequence, loss of proteoglycan and water content deteriorates disc function, and eventually the affected disc degenerates. A source of many significant symptoms, disc degeneration has become the leading etiology of some spinal disorders and low back pain, which inevitably cause considerable socioeconomic issues (3, 4).

Since imbalanced anabolism and catabolism promotes disc degeneration, the biological strategy to increase disc turnover anabolically with growth factors attracts extensive interest. A variety of growth factors such as the bone morphogenetic protein (BMP) family (BMP-2, OP-1/BMP-7, and GDF-5), TGF-β, IGF-1, and FGF have been used to demonstrate their anabolic effects on NP and AF cells in various species (5-12). Furthermore, studies on growth factor receptor expression showed that receptors for these growth factors were expressed by the disc cells at NP and inner AF in both normal and degenerated discs (13). Although it is unknown which growth factor has the best capacity for IVD regeneration, it has been shown that BMP receptor (BMPRII) was the only one not present in blood vessels within the IVD, but was highly expressed in the NP of the severely degenerated disc, suggesting that BMPs did not participate in the undesired blood vessel ingrowth at the IVD and may favor regeneration of degenerated disc (13). BMPs have become promising growth factors and have been the topic of a series of investigations for retarding or even reversing disc degeneration.

BMP-2, the only BMP family member approved for spine surgery by the U.S. Food and Drug Administration to date, has also been extensively evaluated for its potential on IVD regeneration. BMP-2 has been reported to stimulate gene expression in aggrecan and collagen type II in cultured rat IVD cells (7, 10). More importantly, a study by Kim et al. found that in cultured human degenerative IVD cells, BMP-2 treatment facilitated expression of a chondrogenic phenotype of human IVD cells and no evidence of bone nodule formation was observed (14). All together, these findings demonstrated that BMP-2 elicits IVD anabolism by stimulating matrix synthesis of IVD cells. However, because of the high cost and relatively brief half-life of BMP-2, its clinical application to degenerative disc disease is currently economically unfeasible. Moreover, to obtain its effectiveness, high exogenous concentration has to be delivered and, thus, cyto- and physio-tolerance is a concern. It is important to look for stable, small molecules that can stimulate BMP-2 expression and, in turn, promote anabolic metabolism of the IVD cells, resulting in disc matrix regeneration.

A recent study indicated that 3-hydroxy-3-methylglutaryl coenzyme A (HMG-CoA) reductase inhibitor statins increase BMP-2 mRNA in murine and human bone cells in vitro, and subsequent bone formation in vivo (15). Statins are the commonly prescribed cholesterol-lowering drugs that can inhibit the pathway of cholesterol biosynthesis. Further studies demonstrated that statins increase BMP-2 expression in various types of cells such as non-transformed osteoblastic cells, bone marrow stromal cells, human vascular smooth muscle cells, and rat chondrocytes (16-19). However, there has been no effort to date to evaluate whether statins can also increase BMP-2 expression and, in turn, stimulate matrix synthesis of IVD cells.

Thus, there remains a need in the art to develop an effective method to treat cartilage degeneration, such as that seen in spinal disc and other joints.

### SUMMARY OF THE INVENTION

The present invention relates to a method for treating damage at a site of cartilage injury comprising administering a composition that increases expression of bone-morphogenic proteins, such that the composition is administered in a controlled release formulation and delivered at or near the site of injury.

In one aspect, the invention contemplates a method to repair or retard cartilage damage comprising the step of administering to a subject in need thereof a composition that increases bone morphogenic protein (BMP) expression directly into a site of cartilage injury in a controlled release formulation, wherein said composition is released in said cartilage injury at a rate and an amount effective to permit repair of or retard said damage.

In a related aspect, the invention contemplates a method to repair or retard cartilage damage comprising the step of administering to a subject in need thereof a statin that increases bone morphogenic protein expression to a site of cartilage injury in a controlled release formulation, wherein said statin is released in said cartilage injury at a rate and an amount effective to permit repair of or retard said damage.

In one embodiment, the cartilage is in a spinal disc. In a related embodiment, the cartilage is in a joint.

In a further embodiment, the joint is selected from the group consisting of a fibrous joint, a cartilaginous joint and a synovial joint. The joint in the method is any joint known in the art and susceptible to cartilage damage, either through trauma to the site or due to natural or degenerative degradation of the cartilage over time. In one embodiment, the joint is a knee joint. In another embodiment, the joint is a ball and socket joint.

In another embodiment, the administering is by injection. In further embodiments, the administration is via depot or implant of a vessel comprising a composition that increases BMP expression.

In a related embodiment, the injecting is carried out using a fluoroscope to guide a syringe carrying the controlled release formulation. In some embodiments, the syringe is maintained at 4° C. and the controlled release formulation is in a fluid state.

In one embodiment, the present invention provides methods to repair or retard spinal disc damage comprising the step of injecting a composition that increases bone morphogenic protein expression directly into an injured disc in a controlled release formulation, wherein said composition is released in said disc injury at a rate and an amount effective to permit repair of said damage.

The present invention provides methods to repair or retard spinal disc damage comprising the step of injecting a statin that increases bone morphogenic protein expression directly into an injured disc in a controlled release formulation, wherein said statin is released in said disc injury at a rate and an amount effective to permit repair of said damage.

In a further embodiment, the cartilage injury shows an initial indication of damage and said method repairs said initial damage or retards additional damage.

In one embodiment, the BMP is any BMP known in the art. BMPs include, but are not limited to, BMP-1, BMP-2, BMP-3, BMP-4, BMP-5, BMP-6, BMP-7, BMP-8a and BMP-9. In certain embodiments, the BMP is selected from the group consisting of BMP-2 and BMP-7.

In a further embodiment, the composition is selected from the group consisting of a statin or derivative thereof, an isoprenyl transferase inhibitor, BMP-2 and a BMP activating agent.

In various embodiments of the invention, the statin is selected from the group consisting of lovastatin, mevastatin, pravastatin, pitavastatin, simvastatin, atorvastatin, cerivastatin, fluvastatin, and rosurvastatin, as described in Choudhury, et al., J. Biol. Chem., 282(7):4983-4993, 2007; Ohnaka, et al., Biochem Biophys Res Comm, 287(2):337-342, 2001; and Veillard and Mach, Cell Mol Life Sci. 59(11):1771-86, 2002. In one embodiment, the statin is simvastatin.

In a related embodiment, the BMP activating agent is a statin intermediate. Exemplary statin intermediates include, but are not limited to, molecules involved in the pathway of statin activation of a BMP, such as BMP-2; and protein regulation downstream of BMP signaling; homeodomain proteins, e.g., MSX2, DLXD3, and DLX5, transcription factors, e.g., RUNX2 (CBFA1/AML3), and an SP1 family member OSTERIX); SMADs; cGMP; MAP kinase; p38; JNK; alkaline phosphatase and osteocalcin.

In a further embodiment, the isoprenyl transferase inhibitor is selected from the group consisting of benzodiazepine and derivatives thereof, manumycin A, FTI-254, FTI-277, FTI-2153, SCH66336, SCH 44342, BZA-5B, R115777, BMS-214662, perillic acid, GGTI-286 and GGTI-298.

In some embodiments, the controlled release formulation is a hydrogel. In one embodiment, the hydrogel is a temperature sensitive hydrogel. In a related embodiment, the hydrogel is a pH sensitive hydrogel. In a further embodiment, the hydrogel comprises a hydrophobic polymer and a hydrophilic polymer.

In certain embodiments, the invention provides that the hydrophilic polymer in the hydrogel is selected from the group consisting of polyethylene glycol (PEG), poly(ethylene oxide) (PEO), polyoxyethylene (POE), polyvinyl alcohols, hydroxyethyl celluloses, dextrans, and combinations thereof. In a further embodiment, the hydrophobic polymer in the hydrogel is selected from the group consisting of poly lactic acid (PLA), polyglycolic acid poly(lactide-co-glycolide) (PLGA), poly(ε-caprolactone), (PCL), polyanhydrides, polyesters, polyorthoesters, polyetheresters, polycaprolactone, polyesteramides, polycarbonate, polycyanoacrylate, polyurethanes, polyacrylate, and combinations thereof. In various embodiments, the polymers are homopolymers or copolymers.

In one embodiment, the hydrogel composition is selected from the group consisting of PLGA-PEG-PLGA, PLA-PEG-PLA, PEG-PLGA-PEG, and PEG-PLA-PEG.

In various embodiments, the molecular weight of the PEG is from about 3 kDa to about 200 kDa, from about 5 to about 120 kDa, from about 10 to about 100 kDa, from about 20 to about 50 kDa, from about 5 kDa to about 60 kDa, from about 5 kDa to about 40 kDa, from about 3 to about 30 kDa, from about 5 kDa to about 25 kDa, from about 5 kDa to about 15 kDa, from about 5 kDa to about 10 kDa, or from about 0.5 to about 10 kDa. In one embodiment, is contemplated that the PEG is about 0.5 kDa, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa. In other embodiments, the PEG is about 15 kDa, about 20 kDa, about 25 kDa, is about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 110 kDa, about 120 kDa, about 130 kDa, about 140 kDa, about 150 kDa, about 160 kDa, about 170 kDa, about 180 kDa, about 190 kDa, or about 200 kDa.

In a related embodiment, the PEG has a molecular weight selected from the group consisting of about 0.5 kDa, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa.

In one embodiment, the controlled release formulation is a hydrogel comprising PLGA-PEG-PLGA polymers and a statin.

In certain embodiments, it is contemplated that administering the controlled release composition promotes proliferation of chondrocytes or chondrocyte-like cells in the damaged cartilage site. In a related embodiment, administering the controlled release composition promotes expression of collagen II and aggrecan in the damaged cartilage site.

In various embodiments, the subject in need is a mammal. In one embodiment, the subject is human.

Also, in other aspects of methods of the invention, the controlled release formulation includes a component to control release as described herein.

In one aspect, the invention provides a controlled release formulation for administering directly into damaged cartilage comprising a composition that increases bone morphogenic protein (BMP) expression, wherein administration of the controlled release formulation into the cartilage provides the compound at a rate and an amount effective to permit repair of, to retard damage to or to prevent additional damage to said cartilage.

In a related aspect, the invention contemplates a controlled release formulation for administering directly into damaged cartilage comprising a statin that increases bone morphogenic protein (BMP) expression, wherein administration of the controlled release formulation into the cartilage provides the statin at a rate and an amount effective to permit repair of, to retard damage to or to prevent additional damage to said cartilage.

In one embodiment, the cartilage is in a spinal disc. In a related aspect, the cartilage is in a joint. In a further embodiment, the joint is selected from the group consisting of a fibrous joint, a cartilaginous joint and a synovial joint. The joint is any joint known in the art that is susceptible to cartilage damage. In some embodiments, the joint is a knee joint. In further embodiments, the joint is a ball and socket joint.

In one embodiment, the BMP is any BMP known in the art. BMPs include, but are not limited to, BMP-1, BMP-2, BMP-3, BMP-4, BMP-5, BMP-6, BMP-7, BMP-8a and BMP-9. In certain embodiments, the BMP is selected from the group consisting of BMP-2 and BMP-7.

In various embodiments, the administering is by injection. In a related embodiment, the injection is carried out using a fluoroscope to guide a syringe carrying the controlled release formulation. In a further embodiment, the syringe is maintained at 4° C. and the controlled release formulation is in a fluid state.

In certain embodiments, the composition in the controlled release formulation is selected from the group consisting of a statin or derivative thereof, an isoprenyl transferase inhibitor, BMP-2 and a BMP activating agent.

In one embodiment, the statin is selected from the group consisting of lovastatin, mevastatin, pravastatin, simvastatin, atorvastatin, cerivastatin, fluvastatin, rosurvastatin. In a related embodiment, the statin is simvastatin.

In one embodiment, the BMP activating agent in the controlled release formulation is a statin intermediate. Exemplary statin intermediates include, but are not limited to, molecules involved in the pathway of statin activation of a BMP, such as BMP-2, and protein regulation downstream of BMP signaling; homeodomain proteins, e.g., MSX2, DLXD3, and DLX5, transcription factors, e.g., RUNX2 (CBFA1/AML3), and an SP1 family member OSTERIX); SMADs; cGMP; MAP kinase; p38; JNK; alkaline phosphatase and osteocalcin.

In another embodiment, the isoprenyl transferase inhibitor is selected from the group consisting of benzodiazepine and derivatives thereof, manumycin A, FTI-254, FTI-277, FTI-2153, SCH66336, SCH 44342, BZA-5B, R115777, BMS-214662, perillic acid, GGTI-286 and GGTI-298.

In some embodiments, the controlled release formulation is a hydrogel. In one embodiment, the hydrogel is a temperature sensitive hydrogel. In a related embodiment, the hydrogel is a pH sensitive hydrogel. In a further embodiment, the hydrogel comprises a hydrophobic polymer and a hydrophilic polymer. It is contemplated that the hydrophilic polymer and hydrophobic polymer in the controlled release formulation are as described above and elsewhere herein. It is further contemplated that the hydrophobic polymers and hydrophilic polymers are homopolymers or copolymers.

In one embodiment, the hydrogel in the controlled release formulation is selected from the group consisting of PLGA-PEG-PLGA, PLA-PEG-PLA, PEG-PLGA-PEG, and PEG-PLA-PEG.

In one embodiment, the molecular weight of the PEG is in a range from about 3 kDa to about 200 kDa, from about 5 to about 120 kDa, from about 10 to about 100 kDa, from about 20 to about 50 kDa, from about 5 kDa to about 60 kDa, from about 5 kDa to about 40 kDa, from about 3 to about 30 kDa, from about 5 kDa to about 25 kDa, from about 5 kDa to about 15 kDa, from about 5 kDa to about 10 kDa, or from about 0.5 to about 10 kDa. In one embodiment, is contemplated that the PEG is about 0.5 kDa, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa. In other embodiments, the PEG is about 15 kDa, about 20 kDa, about 25 kDa, is about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 110 kDa, about 120 kDa, about 130 kDa, about 140 kDa, about 150 kDa, about 160 kDa, about 170 kDa, about 180 kDa, about 190 kDa, or about 200 kDa.

In related embodiments, the PEG has a molecular weight selected from the group consisting of about 0.5 kDa, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa.

In certain embodiments, the hydrophilic polymer in the hydrogel is in a range from about 10% to about 50%, from about 20% to about 40%, or from about 20% to about 30%. In one embodiment, the hydrophilic polymer is in a range of about 20% to about 30%.

In other embodiments, the hydrophobic polymer in the hydrogel is in a range from 40% to about 90%, from about 60% to about 80%, or from about 70% to about 80%. In one embodiment, the hydrophobic polymer is in a range of about 70% to about 80%.

In one embodiment, the composition in the hydrogel is in a range from about 1 to about 50 mg/ml, from about 5 to about 30 mg/ml, from about 5 to about 25 mg/ml, from about 5 to about 20 mg/ml, from about 10 to about 30 mg/ml, from about 1 to about 15 mg/ml, from about 5 to about 15 mg/ml, or any amount determined by one of ordinary skill to be appropriate for the hydrogel formulation and the composition solubilized in the hydrogel.

In one embodiment, the controlled release formulation is a hydrogel comprising PLGA-PEG-PLGA polymers and a statin.

In another aspect, the invention provides a method for delivering a composition that increases bone morphogenic protein (BMP) expression directly into damaged cartilage in a controlled release formulation to a patient in need thereof comprising, administering the composition in the controlled release formulation through a syringe into a site of cartilage injury, wherein the syringe is guided to the site of injury using a fluoroscope, and wherein administration of the controlled release formulation into the cartilage provides the composition at a rate and an amount effective to permit repair of, to retard damage to or to prevent additional damage to said cartilage.

In one embodiment, the site of cartilage injury is an intervertebral disc and the administering comprises, placing a radiolucent marker on skin of the patient to identify a point of entry for percutaneous access to the disc; fluoroscopically guiding a spinal needle containing the composition in a controlled release formulation into the disc posterolaterally, and optionally injecting a radio-opaque tracer into the disc to determine placement and integrity of the disc.

In a related embodiment, the syringe enters the disc by an extrapedicular approach through an access triangle delineated by a superior articular process as described herein.

Other features and advantages of the invention will become apparent from the following detailed description. It should be understood, however, that the detailed description and the specific examples are given by way of illustration only. Various changes and modifications within the spirit and scope of the invention will become apparent to those skilled in the art from this detailed description.

### BRIEF DESCRIPTION OF THE DRAWINGS

FIG. 1 shows the effects of simvastatin on aggrecan mRNA expression in three dimensional (3D) culture over time and at varying concentrations of simvastatin.

FIG. 2 shows the effects of simvastatin on collagen II mRNA expression in 3D culture over time and at varying concentrations of simvastatin.

FIG. 3 shows the effect of culture time on aggrecan and collagen type II mRNA expression in rat IVD cells cultured in alginate beads. Data are normalized with internal control (GAPDH) and are expressed as ratio to Day 0.

FIG. 4 shows the effect of simvastatin on BMP-2 mRNA (FIG. 4A) and protein (FIG. 4B) expression in rat IVD cells cultured in alginate beads. Data are normalized with internal control (GAPDH) and are expressed as ratio to Vehicle (*, P<0.05 and **, P<0.01 compared with Vehicle). Simvastatin increased BMP-2 mRNA expression in a dose dependent manner and the level reached the maximum at day 2.

FIG. 5 shows the effect of simvastatin on aggrecan and collagen type II mRNA expression in rat IVD cells cultured in alginate beads. Data are normalized with internal control (GAPDH) and are expressed as ratio to Vehicle (*, P<0.05 and **, P<0.01 compared with Vehicle). Simvastatin significantly increased aggrecan and collagen type II mRNA expression and both expressions reached the maximum at day 21.

FIG. 6 shows the effect of simvastatin on sGAG production in rat IVD cells cultured in alginate beads. Data are normalized with cell DNA content and are expressed as ratio to Vehicle (*, P<0.05 and **, P<0.01 compared with Vehicle). Simvastatin increased sGAG content significantly at day 7 and after.

FIG. 7 shows the effects of noggin (A) 0.5 μg/ml, day 7 (B) 3 μg/ml, day 7 and (C) 3 μg/ml, day 14 on simvastatin (3 μM) activity on aggrecan and collagen type II mRNA expression in rat IVD cells cultured in alginate beads. Data are normalized with internal control (GAPDH) and are expressed as ratio to Veh+Veh (*, P<0.05 and **, P<0.01 compared with Veh+Veh; #, P<0.05 and ##, P<0.01 compared with Veh+S3). Noggin (500 ng/ml) significantly blocked both upregulated gene expression induced by 3 μM of simvastatin at day 7, but not day 14. Noggin at 3 μg/ml attenuated upregulated type II collagen gene expression at day 14.

FIG. 8 shows the effect of mevalonate (200 μM) on simvastatin (3 μM) activity on BMP-2, aggrecan, and collagen type II mRNA expression in rat IVD cells cultured in alginate beads. Data are normalized with internal control (GAPDH) and are expressed as ratio to Veh+Veh (*, P<0.05 and **, P<0.01 compared with Veh+Veh; #, P<0.05 and ##, P<0.01 compared with Veh+S3). Mevalonate completely reversed upregulated gene expression by simvastatin at day 7 and day 14.

FIG. 9 shows the effect of simvastatin on cell viability in rat IVD cells. Data are expressed as ratio to Vehicle (*, P<0.05 and **, P<0.01 compared with Vehicle). Simvastatin dose- and time-dependently decreased IVD cell viability.

FIG. 10 illustrates the effect of simvastatin on the mRNA expression of BMP-2 of the human NP and AF cells. Data are normalized with GAPDH and are expressed as ratio to Vehicle (*, P<0.05 and **, P<0.01 compared with vehicle).

FIG. 11 depicts the effects of simvastatin on aggrecan (A), collagen type II (B), collagen type I (C) mRNA expression and the “differentiation index” collagen II/I ratio of the human NP and AF cells (D). Data are normalized with GAPDH and are expressed as ratio to Vehicle (*, P<0.05 and **, P<0.01 compared with Vehicle).

FIG. 12 illustrates a needle stab into the intervertebral disc to create injury that induces later disc degeneration by the guidance of fluoroscopy.

FIG. 13 depicts the T2-weighted Magnetic Resonance images at the perturbed and the normal disc levels at 4 weeks (A), and 8 weeks (B) after the stab injury.

FIG. 14 illustrates the changes of the T2-weighted image intensity (B, D) and the MRI index (A,C) at 4 weeks (A,B), and 8 weeks (C,D) after the stab injury (MRI index=the number of pixels×the corresponding image densities). *, p<0.05 and **, p<0.01 compared to Control group.

FIG. 15 shows the T2-weighted magnetic resonance images at the polymer injected and the normal disc levels at 2 weeks after the injection.

FIG. 16 shows that the injection of simvastatin-loaded polymer reversed the degenerative discs back to the normal intensity level (B) and the MRI index (A) 2 weeks after the injection. *, p<0.05 compared to control group; #, p<0.05 compared to polymer alone.

FIG. 17 is a comparison of the wet weight of NP among different treated and untreated groups. Note that the injection of the simvastatin-loaded polymer (5 mg/ml, inject volume: 2 μl) retained the wet weight in the degenerated discs at 2 weeks after the injection (**, p<0.01 compared to the intact control group).

### DETAILED DESCRIPTION

The present invention is directed to methods for repairing or retarding vertebral disc degeneration in a mammal comprising administering a controlled-release composition comprising a statin or other BMP-activating agent. In one aspect, the controlled release composition is a hydrogel comprising a water soluble polymer.

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

As used herein, the following terms have the meanings ascribed to them unless specified otherwise.

As used herein, “to repair or retard cartilage damage” refers to a method to treat a site of injury or damage in an area of cartilage. Damage refers to a tear, abrasion, lesion or other aberration or wound in the cartilage structure in a subject compared to normal, non-damaged cartilage. In some embodiments, damage arises from a trauma or strike to the cartilage or areas surrounding the cartilage such that the cartilage is damaged. In other embodiments, the cartilage damage arises from degeneration of the cartilage due to normal degradation over time or due to a degenerative disorder. To repair cartilage damage is to partially or completely heal the wound in the cartilage. To retard damage is to curtail or slow the initial damage from progressing in the cartilage, thereby preventing further damage. To prevent further damage is to slow or stop the progression of damage in the cartilage such that no significant additional damage is observed in the cartilage site.

As used herein, a “composition that increases bone morphogenic protein (BMP) expression” or “a BMP-activating agent” refers to a composition that increases either the mRNA expression of a BMP gene, increases protein expression of a BMP and/or increases BMP protein activity. BMPs play a role in bone and cartilage formation. Exemplary BMPs include, but are not limited to BMP-1, BMP-2, BMP-3, BMP-4, BMP-5, BMP-6, BMP-7, BMP-8a and BMP-9.

As used herein, the term “statin” refers to a class of HMG-CoA reductase inhibitors typically used in methods to lower cholesterol. Exemplary statins include, but are not limited to, lovastatin, mevastatin, pravastatin, pitavastatin, simvastatin, atorvastatin, cerivastatin, fluvastatin, and rosurvastatin.

As use herein, a “statin intermediate” refers to a compound in the biochemical pathway of statin signaling that results in upregulation of bone morphogenic protein expression in a cell.

As used herein, an “isoprenyl transferase inhibitor” refers to a compound that inhibits transferases involved in adding an isoprenyl group to the N- or C-terminus of a protein. Isoprenyl transferases include geranylgeranyl protein transferase I and II, (GGPTase I or GGPTase II) and farnesyl protein transferase (FPTase). Isoprenylated intermediates are necessary in the cholesterol biosynthetic pathway, and a decrease in isoprenylation decreases cholesterol synthesis, similar to the effect of statin.

Exemplary farnesyl transferase inhibitors include, but are not limited to benzodiazepine and derivatives thereof, manumycin A, FTI-254, FTI-277, FTI-2153, SCH66336, SCH 44342, BZA-5B, R115777 and BMS-214662. Exemplary geranylgeranyl transferase inhibitors include, but are not limited to, perillic acid, GGTI-286 and GGTI-298. Additional inhibitors of isoprenyl transferases are described, for example, in U.S. Pat. Nos. 6,486,202 and 6,376,468, and Bishop et al., (J Biol Chem 270: 30611-30618, 1995).

As used herein, a “BMP-2 activating agent” is a compound that activates expression, either mRNA an protein expression, of BMP-2, and/or BMP-2 protein activity. In various embodiments, it is contemplated that a BMP-2-activating agent is a protein, peptide, peptidomimetic, or small molecule. In one aspect, BMP-2 activating agents are identified using chemical libraries, antibody libraries, and other methods known in the art to identify molecules that stimulate activity of a protein.

As used herein, a “sustained-release” or “controlled-release” formulation comprises an active agent and a substance for extending the physical release or biological availability of the active agents over a desired period of time. In one aspect, the controlled release formulation comprises a statin compound or other BMP-activating agents, and a sustained-release substance for extending the physical release or biological availability of the statin or other BMP-activating agents over a desired period of time. Controlled release formulations are described in further detail herein.

In one aspect, it is contemplated that the controlled release formulation is a hydrogel. A “hydrogel” refers to a controlled release formulation comprising a combination of hydrophilic and hydrophobic block polymers.

As used herein, “polymer molecule” or “physiologically acceptable polymer molecule” refers to polymer molecules which are substantially soluble in aqueous solution or may be present in form of a suspension and have substantially no negative impact to mammals upon administration of a polymer-protein conjugate in a pharmaceutically effective amount and are regarded as biocompatible. In one embodiment, physiologically acceptable molecules comprise from about 2 to about 1000, about 2 to about 900, about 2 to about 800, about 2 to about 700, about 2 to about 600, about 2 to about 500, about 2 to about 400, about 2 to about 300, about 2 to about 200, or from about 2 to about 100 repeating units. Exemplary physiologically acceptable polymers include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphasphazene, polyoxazoline, poly(N-acryloylmorpholine), poly(alkylene oxide) polymers, poly(maleic acid), poly(DL-alanine), polysaccharides, such as carboxymethylcellulose, dextran, hyaluronic acid and chitin, poly(meth)acrylates, and combinations of any of the foregoing.

The polymer molecule is not limited to a particular structure and, in certain aspects, is linear (e.g. alkoxy PEG or bifunctional PEG), branched or multi-armed (e.g. forked PEG or PEG attached to a polyol core), dendritic, with or without degradable linkages. Moreover, the internal structure of the polymer molecule are, in still other aspects, organized in any number of different patterns and are selected from the group consisting of, without limitation, homopolymer, alternating copolymer, random copolymer, block copolymer, alternating tripolymer, random tripolymer, and block tripolymer.

“Pharmaceutical composition” refers to a composition suitable for pharmaceutical use in subject animal, including humans and mammals. A pharmaceutical composition comprises a pharmacologically effective amount of a polymer-polypeptide conjugate and also comprises a pharmaceutically acceptable carrier. A pharmaceutical composition encompasses a composition comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound or conjugate of the present invention and a pharmaceutically acceptable carrier.

“Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration). A “pharmaceutically acceptable salt” is a salt that is formulated into a compound or conjugate for pharmaceutical use including, e.g., metal salts (sodium, potassium, magnesium, calcium, etc.) and salts of ammonia or organic amines.

“Pharmaceutically acceptable” refers to a material which is not biologically or otherwise undesirable, i.e., the material may be administered to an individual without causing any undesirable biological effects or interacting in a deleterious manner with any of the components of the composition in which it is contained.

### Statins, Derivatives Thereof and Other BMP-Activating Agents

Statins are a group of related compositions that block the activity of HMG-CoA reductase and interfere with the cholesterol synthesis pathway. Statins have been used as therapeutics to reduce high cholesterol in patients. Statins have also been shown to have a pleotropic effect in vivo, demonstrating involvement in regulation of inflammation (Grip et al., Br J Pharmacol. 155(7):1085-92, 2008), and aiding in decreasing amyloid β deposition in Alzheimers Disease ((Ostrowski et al., J Biol Chem. 282(37):26832-44, 2007; Cordle et al., J Biol Chem. 280(40):34202-9, 2005). Simvastatin has been observed to increase expression of BMP-2 and in turn stimulate bone formation (Mundy G. et al., Science 286(5446): 1946-9, 1999) and chondrogenic phenotype (Hatano H. et al., J Orthop Sci, 8(6): 842-8, 2003).

Statins include, for example and without limitation, ovastatin, mevastatin, pravastatin, pitavastatin, simvastatin, atorvastatin, cerivastatin, fluvastatin, and rosurvastatin.

Statin derivatives are chemical derivatives of the statins that demonstrate the same or similar activity but have a different chemical composition. Statin derivative solistatin (VIII) is disclosed by in PCT publication No. WO 03/048148. U.S. Pat. No. 4,255,444 teaches statin derivatives that are 4-hydroxy-2-pyrone compounds. Additional statin derivatives contemplated for use in the invention, include but are not limited to, those described in, for example, WO/2006/123355, U.S. Pat. No. 5,134,124, Ennio et al. (Proc Natl Acad Sci USA 2004; 101(22):8497-502, 2004, directed to NO-releasing statin derivatives such as NCX 6550 and NCX 6553), and other statin derivatives known (or after-discovered) and disclosed in the art.

Addition of an isoprenyl group onto certain proteins is required for completion of the cholesterol synthesis, as well as activation of certain common signaling pathways, including the Ras signaling pathway. Addition of isoprenyl groups is carried out by three different isoprenyl transferases, farnesyl protein transferase (FTPase), and geranylgeranyl protein transferase I and II (GGTPase I and GGTPase II). Statins have been shown to inhibit isoprenylation of proteins in cholesterol synthesis as well as in other cellular reactions (Ostrowski et al., J Biol Chem. 282(37):26832-44, 2007; Cordle et al., J Biol Chem. 280(40):34202-9, 2005). Isoprenyl transferase inhibitors inhibit the transfer of an isoprenyl group to the N- or C-terminus of a protein, thereby inhibiting various signaling pathways, and isoprenyl transferase inhibitors have also been shown to have similar properties as statins in inhibition of cholesterol. As such, agents that simulate the effects of statins in vitro include isoprenyl transferase inhibitors. It is contemplated that isoprenyl transferase inhibitors are useful in the methods and compositions of the invention to retard or repair cartilage degeneration in a subject.

In one embodiment, the isoprenyl transferase inhibitor is a farnesyl transferase inhibitor (FTI) or a geranylgeranyl transferase inhibitor (GGTI). Exemplary farnesyl transferase inhibitors include, but are not limited to benzodiazepine and derivatives thereof, manumycin A, FTI-254, FTI-277, FTI-2153, SCH66336, SCH 44342, BZA-5B, R115777 and BMS-214662. Exemplary geranylgeranyl transferase inhibitors include, but are not limited to, perillic acid, GGTI-286 and GGTI-298. Additional inhibitors of isoprenyl transferases are described, for example, in U.S. Pat. Nos. 6,486,202 and 6,376,468, Bishop et al., (J Biol Chem 270: 30611-30618, 1995). In various embodiments, the isoprenyl transferase inhibitor is selected from the group consisting of benzodiazepine and derivatives thereof, manumycin A, FTI-254, FTI-277, FTI-2153, SCH66336, SCH 44342, BZA-5B, R115777, BMS-214662, perillic acid, GGTI-286 and GGTI-298.

BMP-activating agents include proteins, peptides, peptidomimetics, small molecules, and other compounds that induce expression of BMP mRNA or protein. In one aspect, BMP-activating agents are identified using chemical libraries, antibody libraries, and other methods known in the art to identify molecules that stimulate activity of a protein. Exemplary BMP-2-activating agents, include but are not limited to, B2A2, a synthetic peptide having BMP-2 activating effects (Lin et al., J Bone Miner Res. 20(4):693-703, 2005), and a synthetic peptide corresponding to BMP-2 residues 73-92, shown to induce osteogenic effects in vitro (Saito et al., 1: J Biomed Mater Res A. 72(1):77-82, 2005).

Statin intermediates refers to molecules involved in the pathway of statin activation of BMP, and protein regulation downstream of BMP signaling. For example, it has been demonstrated that homeodomain proteins MSX2, DLXD3, and DLX5 are activated in the osteogenic pathway in response to BMP-2 (Hassan et al., J Biol Chem. 281(52):40515-26, 2006). Additional statin intermediates in the BMP signaling pathway include transcription factors (such as RUNX2 (CBFA1/AML3), and an SP1 family member OSTERIX), SMADs, and cGMP. Additional factors involved in BMP-2 signaling pathways include MAP kinase, p38, JNK, alkaline phosphatase and osteocalcin (Guicheux et al., J Bone Miner Res. 18(11):2060-8, 2003).

### Controlled Release Formulations

Sustained-release or “controlled-release” formulations of statin compounds or other BMP activating agents, include a statin or other BMP activating agents and a sustained-release substance for extending the physical release or biological availability of the statin or other BMP activating agents over a desired period of time. Sustained-release forms include, but are not limited to, statin or other BMP-activating agents compounds encapsulated in sustained-release means such as a slowly-dissolving biocompatible polymer (for example, the polymeric carriers known in the art, alginate microparticles as described in U.S. Pat. No. 6,036,978, or polyethylene-vinyl acetate and poly(lactic-glucolic acid) compositions described in U.S. Pat. No. 6,083,534), admixed with such a polymer (including for example and without limitation hydrogels), and or encased in a biocompatible semi-permeable implant. Additional sustained-release forms also include injectable polymeric microparticles, wherein a statin and for example, a polymer are mixed, and lyophilized to in a process that forms microparticles as described in U.S. Pat. No. 6,020,004; injectable gel compositions comprising a biodegradable anionic polysaccharide such as an alginate ester, a polypeptide, and at least one bound polyvalent metal ion as described in U.S. Pat. No. 6,432,449; injectable biodegradable polymeric matrices having reverse thermal gelation properties and optionally pH-responsive gelation/de-gelation properties (U.S. Pat. Nos. 6,541,033 and 6,451,346); and biocompatible polyol:oil suspensions comprising, for example, polyol in the range of from about 15% to about 30% by weight (U.S. Pat. No. 6,245,740).

Additional statin sustained- or controlled-release forms include those described in Kim, C., 2000, “Controlled Release Dosage Form Design”, Techonomic Publishing Co., Lancaster Pa., such as natural polymers (gelatin, sodium alginic acid, xanthan gum, arabic gum, or chitosan), semi-synthetic polymers or cellulose derivatives (methylcellulose, ethylcellulose, hydroxyethylcellulose, hydroxypropylcellulose, hydroxyethylmethylcellulose, hydroxypropylmethylcellulose, sodium carboxymethylcellulose, cellulose acetate, cellulose acetate butyrate, cellulose acetate proprionate, cellulose acetatephthalate, or hydroxypropylmethylcellulose phthalate), and synthetic polymers (ion exchange resins (methacrylic acid, sulfonated polystyrene/divinylbenzene), polyacrylic acid (Carbopol), poly(MMA/MAA), poly(MMA/DEAMA), poly(MMA/EA), poly(vinylacetate phthalate), poly(vinyl alcohol), poly(vinyl pyrrolidone), poly(lactic acid), poly(glycolic acid), poly(lactic/glycolic acid), polyethylene glycol, polyethylene oxide, poly(dimethyl silicone), poly(hydroxyethyl methacrylate), poly(ethylene/vinyl acetate), poly(ethylene/vinyl alcohol), polybutadiene, poly(anhydride), poly(orthoester), and poly(glutamic acid)). Additional statin compositions for sustained- or controlled-delivery include liposome carriers (Eppstein et al., Proc. Natl. Acad. Sci. (USA), 82:3688-3692 (1985); EP 36,676; EP 88,046; EP 143,949), biodegradable porous beads (PCT/US93/00829), semipermeable polymer matrices including, e.g. films and microcapsules, matrices such as polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277, (1981) and Langer et al., Chem. Tech., 12:98-105(1982)), ethylene vinyl acetate (Langer et al., supra) or poly-D(−)-3-hydroxybutyric acid (EP 133,988).

### Thermosensitive Polymers

When a statin or other BMP-activating compound is incorporated into an appropriate carrier, the exposure of statin or other compound to the intervertebral disc cells or other site of cartilage injury is, in one aspect, confined within the discal core or the joint site and the delivery is sustained. For this purpose, a thermo-sensitive, biodegradable block copolymer is useful for local drug delivery. Studies using a thermosensitive polymer as a drug delivery system are found, for example, (Jeong et al., Nature 388(6645): 860-862, 1997; Zentner et al., J Controlled Release 72(1-3): 203-215, 2001) and its biodegradability (Jeong et al., J Biomed Mater Res 50(2): 171-177, 2000), which describe sol-gel phase transition properties as a function of temperature.

In one embodiment, the thermosensitive polymer is a hydrogel. In some aspects, the hydrogel contemplated for use in the invention comprises utilization of block copolymers having biodegradable hydrophobic block (polymer) segments and hydrophilic block (polymer) segments. In some embodiments, the copolymers are di block, e.g., hydrophobic-hydrophilic, or tri-block copolymers, e.g., hydrophobic-hydrophilic-hydrophobic or hydrophilic-hydrophobic-hydrophilic, type block copolymers. See e.g., U.S. Pat. No. 6,451,346.

Biodegradable hydrophobic block segments contemplated for use include, but are not limited to, poly(α-hydroxy acid) members derived from or selected from the group consisting of homopolymers and copolymers of poly(lactide)s (D- or L-forms), poly(glycolide)s, poly-lactide glycolic acid, polyanhydrides, polyesters, polyorthoesters, polyetheresters, polycaprolactone, polyesteramides, polycarbonate, polycyanoacrylate, polyurethanes, polyacrylate, and combinations thereof.

The range of molecular weights contemplated for the hydrophobic block polymers to be used in the present processes can be readily determined by a person skilled in the art based upon such factors the desired polymer degradation rate. Typically, the range of molecular weight for the hydrophobic block will be 1000 to 20,000 Daltons.

PLGA as used herein refers to a polymer of lactic acid alone, a polymer of glycolic acid alone, a mixture of such polymers, a copolymer of glycolic acid and lactic acid, a mixture of such copolymers, or a mixture of such polymers and copolymers. In various aspects, the PLGA is non-ionic, e.g., hydroxy-terminated, or ionic, e.g., carboxy-terminated. It is contemplated that the ionizable functional groups are on either one or both ends of the polymer chain, and terminal ionizable groups contemplated for use include any ionizable group having a pKa 3-8, e.g., carboxylic acids, amines, sulfonic acids, ammonium salts.

Hydrophilic block polymers contemplated for use in the hydrogel are described in more detail below. Hydrophilic block polymers contemplated for use include polyethylene glycols having average molecular weights of between about 500 and 10,000. These hydrophilic segments may also contain ionizable groups. In a related embodiment, the hydrogel has a molecular weight of between 1,000 and 50,000, or between 5,000 and 35,000.

The pH of the hydrogel is generally about 6.5 to about 7.8, which is a suitable pH level range for injection into the body. The pH level is adjusted by use of any suitable acid or base, such as hydrochloric acid or sodium hydroxide.

Suitable hydrogel polymer solutions contain about 1% to about 80% polymer, about 10% to about 40%, or about 20% to 70% polymer.

In one embodiment of the triblock copolymer hydrogel composition, water comprises more than 70% of the gel mass, which is approximate to the environment of tissues with high water content, such as nucleus pulposus in the intervertedral disc. Moreover, the liquid phase of the injectable polymer (hydrogel) serves as a filling material to enhance damaged tissue properties as well as serving as a drug carriers. In a related embodiment, the polymer retains all its contents upon temperature-induced phase transition, without creating volume changes during the process (i.e., no further swelling or shrinking at body temperature). This is relevant considering the drug-loaded polymer solution is injected into cartilage sites.

In another embodiment, it is contemplated that the polymer simultaneously increases microfibrillar cross-linking of collagen at the annulus fibrosus. In various embodiments, this is carried out by the inclusion of genipin in the polymer formula. Genipin, a gardenia fruit extract, has been found to react with lysine, hydroxylysine, and arginine residues in biological tissues, and thus link these amino acid groups in intramolecular, intermolecular, and microfibrillar bonds. If the polymer does not seal annular tears completely, addition of genipin in the polymer will help seal the tears by the exogenous cross-linking and may in turn increase the functional disc integrity.

Upon increase in temperature above its transition temperature (i.e., body temperature), viscosity increases by approximately 4 orders of magnitude as determined by dynamic mechanical analysis (Zentner et al., supra) and the polymer forms a mechanically stable hydrogel. When injected, the temperature induced phase transition begins immediately to form an outer gel shell, followed by gelation of the rest of the polymer solution within the matter of a few minutes, due to its unique structure and properties in the aqueous solution (Lee, et al., Macromolecular Rapid Communications 22(8): 587-592, 2001). In various aspects, the polymer hydrogel is injectable through a needle tip, including a fine needle tip (i.e., 31 gauge or finer) at 4° C.

The triblock copolymer hydrogel is capable of high drug loading and sustained and controlled drug release. This type of polymer offers delivery of both protein drugs and poorly water-soluble drugs (Zentner et al., supra) due to its unique structure and properties in aqueous solution (Lee et al., supra). The polymer is an amphiphilic block copolymer and is capable of solubilizing poorly water soluble compounds, increasing solubility up to 3 orders of magnitude. Several in vivo studies have demonstrated efficacy in animal models (Zentner et al., supra; Masaki et al., Kidney International 66(5):2061-9, 2004; Samlowski et al., Journal of Immunotherapy 29(5): 524-35, 2006), with different types of drugs (small drugs, hormones, and cytokines) on different target diseases. The rate of drug release can be modulated by manipulating parameters such as initial drug loading and polymer concentration.

Hydrogels have been utilized as implants or depots to administer small molecules and other therapeutics in patients. For example, paclitaxel is approved for administration in a PLGA-PEG comprising hydrogel, and has been the subject of experiments in other hydrogel formulations. See e.g., Shim et al., Int J Pharm. 2007 Feb. 22; 331(1):11-8, 2007; Guo et al., J Biomater Sci Polym Ed. 18(5):489-504, 2007. Use of hydrogels has also been proposed in surgical intervertebral disc and joint replacement surgeries (see e.g., U.S. Patent Publication 20080274161, which describes a hydrogel with a gellant component, and U.S. Patent Publication 20080021563). Block copolymer compositions have also been described, for example, in U.S. Pat. Nos. 6,201,072 and 7,018,645, and U.S. Patent Publications 20080247987, 20070128175 and 20060018872.

In certain embodiments, the controlled release formulation comprises a hydropilic polymer in a range from about 10% to 50%, from about 20% to 40%, or from about 20% to 30%.

In certain embodiments, the controlled release formulation comprises a hydrophobic polymer in a range from about 40% to 90%, from about 60% to 80%, or from about 70%-80%.

In certain embodiments, the controlled release formulation comprises a BMP-activating agent in a concentration up to its maximal loading capacity. In some embodiments, the concentration in the hydrogel is from 1 to 50 mg/ml, from 5 to 30 mg/ml, from 5 to 25 mg/ml, from 5 to 20 mg/ml, from 10 to 30 mg/ml, from 1 to 15 mg/ml, from 5 to 15 mg/ml, or any amount determined by one of ordinary skill to be appropriate for the hydrogel and the composition. For example, the maximal loading capacity for simvastatin in PLGA-PEG-PLGA polymers is 15 mg/ml. One of ordinary skill can readily determine the maximal loading capacity of any of the compositions described herein in the controlled release formulation described herein, using techniques known in the art.

In one aspect of the present study, drug release studies were performed. In an initial investigation for drug loading and release in vitro, 2 mg of simvastatin (SV) was added and stirred overnight at 4° C. per gram of the polymer solution. In a 7-ml glass vial, 1 g of drug-loaded polymer solution was placed, followed by incubation at 37° C. for 15 minutes. Five-milliliters of release medium (10 mM phosphate buffered saline with 2.3% Tween 80 and 10% of PEG 400) at 37° C. was added to each vial and the vials were placed in a shaker bath at 50 r.p.m and the release media were replaced with fresh media at appropriate time points. According to a previous study, simvastatin is a poorly-water soluble drug and its aqueous solubility is about 1.4 μg/ml (Jeon et al., Int J Pharm 340(1-2): 6-12, 2007). Therefore, for these in vitro release studies, sink condition was maintained. That is, the solubility of SV in the release medium was determined to be approximately 500 μg/ml, and the concentration of SV in the medium at any given time point was sufficiently less than 10% of the solubility in the medium. Five mL of the medium was collected at designated time intervals, and analyzed by HPLC. From the in vitro release profile, SV concentration in the vicinity (˜1 c.c.) of the gel-fluid interface was estimated to be greater than 3 μM for greater than 7 days.

In various aspects of the present invention, it is contemplated that the polymer is a block copolymer selected from the group consisting of poly(lactide-co-glycolide)-b-poly(ethyleneglycol)-b-poly(lactide-co-glycolide) (PLGA-PEG-PLGA), PLA-PEG-PLA, PEG-PLA-PEG, PEG-PLGA-PEG, PEG-PCL-PEG, and PCL-PEG-PCL.

It is further contemplated that, in certain aspects, the hydrogels of the invention are pH sensitive hydrogels. See for example, U.S. Patent Publ. 20080293827, and U.S. Pat. No. 7,420,024.

### Hydrophilic Polymers

It is contemplated that the controlled release formulation is a biodegradable hydrogel useful in the method of the invention, comprising a physiologically acceptable polymer molecule, including polymer molecules which, for example, are substantially soluble in an aqueous solution or may be present in form of a suspension and have substantially no negative impact, such as side effects, to mammals upon administration of a hydrogel in a pharmaceutically effective amount and are regarded as biocompatible. There is no particular limitation to the physiologically acceptable polymer molecule used according to the present invention.

The hydrophilic polymer molecules are typically characterized as having for example from about 2 to about 1000, or from about 2 to about 300 repeating units. Examples of such polymer molecules include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphasphazene, polyoxazoline, poly(N-acryloylmorpholine), poly(alkylene oxide) polymers, poly(maleic acid), poly(D,L-alanine), polysaccharides, such as carboxymethylcellulose, dextran, hyaluronic acid and chitin, poly(meth)acrylates, and combinations of any of the foregoing.

For example water-soluble polymers, including but not limited to, poly(ethylene glycol) (PEG), poly(ethylene oxide) (PEO), polyoxyethylene (POE), polyvinyl alcohols, hydroxyethyl celluloses, or dextrans, are contemplated for use in the hydrogel.

PEG, PEO or POE refers to an oligomer or polymer of ethylene oxide. PEGs and PEOs include molecules with a distribution of molecular weights, i.e., polydisperse. The size distribution is characterized statistically by its weight average molecular weight (Mw) and its number average molecular weight (Mn), the ratio of which is called the polydispersity index (Mw/Mn). Mw and Mn are measured, in certain aspects, by mass spectroscopy. Most of the PEG-protein conjugates, particularly those conjugated to PEG larger than 1 KD, exhibit a range of molecular weights due to a polydisperse nature of the parent PEG molecule. For example, in case of mPEG2K (Sunbright ME-020HS, NOF), actual molecular masses are distributed over a range of 1.5˜3.0 KD with a polydispersity index of 1.036. Exceptions are proteins conjugated to MS(PEG)n (n=4, 8, 12 or 24, e.g., PEO4, PEO12)-based reagents (Pierce), which are specially prepared as monodisperse mixtures with discrete chain length and defined molecular weight.

The polymer molecule is not limited to a particular structure and is, in various aspects, linear (e.g. alkoxy PEG or bifunctional PEG), branched or multi-armed (e.g. forked PEG or PEG attached to a polyol core), dentritic, or with degradable linkages. Moreover, the internal structure of the polymer molecule is organized in any number of different patterns and is selected from the group consisting of homopolymer, alternating copolymer, random copolymer, block copolymer, alternating tripolymer, random tripolymer, and block tripolymer.

The invention contemplates PEG-protein conjugates selected from the group consisting of linear PEG-protein conjugates that are NHS-conjugated and range in length from —(CH2-CH2-O)n-, where n=1 to 2000, linear PEG-protein conjugates that are aldehyde-conjugated and range in length from —(CH2-CH2-O)n-, where n=1 to 2000, two-arm branched PEG-protein conjugates that are NHS-conjugated and range in length, from 3 to 100 kDa in mass, and three-arm branched PEG-protein conjugates that are NHS-conjugated. The invention also contemplates hydrogels that contain polymers having different chemical linkages (—CO(CH2)n-, and —(CH2)n- where n=1 to 5) between its conjugation site and the PEG chain. The invention further contemplates charged, anionic PEG to reduce renal clearance, including but not limited to carboxylated, sulfated and phosphorylated compounds (anionic) (Caliceti & Veronese, Adv Drug Deliv Rev 2003 55(10):1261-77; Perlman et al., J Clin Endo Metab 2003 88(7):3227-35; Pitkin et al., Antimicrob Agents Chemother 1986 29(3): 440-44; Vehaskari et al., Kidney Intl 1982 22 127-135).

Macromolecule chemical modification is, in one aspect, performed in a non-specific fashion (leading to mixtures of modified species) or in a site-specific fashion (based on wild-type macromolecule reactivity-directed modification and/or site-selective modification using a combination of site-directed mutagenesis and chemical modification) or, alternatively, using expressed protein ligation methods (Curr Opin Biotechnol. 13(4):297-303 (2002)).

In a further embodiment, the polymer molecules contemplated for use in the hydrogel described herein are selected from among water-soluble polymers or a mixture thereof. In one embodiment, the polymer has a single reactive group, such as an active ester for acylation or an aldehyde for alkylation, so that the degree of polymerization may be controlled. The water-soluble polymer, or mixture thereof if desired, may be selected from the group consisting of, for example, PEG, monomethoxy-PEG, PEO, dextran, poly-(N-vinyl pyrrolidone), propylene glycol homopolymers, fatty acids, a polypropylene oxide/ethylene oxide co-polymer, polyoxyethylated polyols (e.g., glycerol), HPMA, FLEXIMAR™, and polyvinyl alcohol, mono-(C1-C10)alkoxy-PEG, aryloxy-PEG, tresyl monomethoxy PEG, PEG propionaldehyde, bis-succinimidyl carbonate PEG, cellulose, other carbohydrate-based polymers, or mixtures thereof. In certain embodiments, the polymer selected is water-soluble so that the protein to which it is attached does not precipitate in an aqueous environment, such as a physiological environment. The polymer is, in various aspects, branched or unbranched. In one embodiment, for therapeutic use of the end-product preparation, the polymer is pharmaceutically acceptable. Methods for generating peptides comprising a PEG moiety are well-known in the art. See, for example, U.S. Pat. No. 5,824,784.

In one embodiment, the reactive aldehyde is PEG-propionaldehyde, which is water-stable, or mono-C1-C10 alkoxy or aryloxy derivatives thereof (see U.S. Pat. No. 5,252,714). As used herein, PEG is meant to encompass any of the forms of PEG that have been used to derivatize other proteins, such as mono-(C1-C10)alkoxy- or aryloxy-polyethylene glycol.

In one example of the present invention, the hydrogel polymer molecule comprises a PEG and derivatives thereof. There is no specific limitation of the PEG used according to the present invention. For example, PEG-protein conjugates include but are not limited to linear or branched conjugates, multi-armed (e.g. forked PEG or PEG attached to a polyol core), dendritic conjugates, with or without degradable linkages. Moreover, the internal structure of the polymer molecule are, in still other aspects, organized in any number of different patterns and are selected from the group consisting of, without limitation, homopolymer, alternating copolymer, random copolymer, block copolymer, alternating tripolymer, random tripolymer, and block tripolymer. Also contemplated are variants with a different chemical linkage between the PEG chain and conjugation site, and variants differing in lengths. In certain embodiments, the average molecular weight of the PEG ranges from about 3 kiloDalton (“kDa”) to about 200 kDa, from about 5 to about 120 kDa, from about 10 to about 100 kDa, from about 20 to about 50 kDa, from about 5 kDa to about 60 kDa, from about 5 kDa to about 40 kDa, from about 3 to about 30 kDa, from about 5 kDa to about 25 kDa, from about 5 kDa to about 15 kDa, from about 5 kDa to about 10 kDa, or from about 0.5 to about 10 kDa.

In various aspects, it is contemplated that the PEG is about 0.5 kDa, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa. In other embodiments, the PEG is about 15 kDa, about 20 kDa, about 25 kDa, is about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 110 kDa, about 120 kDa, about 130 kDa, about 140 kDa, about 150 kDa, about 160 kDa, about 170 kDa, about 180 kDa, about 190 kDa, or about 200 kDa.

### Pharmaceutical Compositions

In one aspect, the BMP-activating agents useful in the invention are formulated in a pharmaceutical composition comprising one or more pharmaceutically acceptable carriers and admixed with a controlled release formulation. The phrase “pharmaceutically or pharmacologically acceptable” refers to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

Formulation of the pharmaceutical composition will vary according to the route of administration selected (e.g., solution, emulsion). An appropriate composition comprising the composition to be administered is prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

Pharmaceutical compositions useful in the methods of the present invention containing a BMP-activating agent as an active ingredient contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

In some embodiments, the BMP-activating agent compositions are lyophilized for storage and reconstituted in a suitable carrier prior to use. Any suitable lyophilization and reconstitution techniques known in the art are employed. It is appreciated by those skilled in the art that lyophilization and reconstitution leads to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In certain embodiments, the concentration of BMP-activating agent in these formulations varies widely, for example from less than about 0.5%, usually at or at least about 1% to as much as 15 or 20% by weight and will be selected primarily based on fluid volumes, viscosities, etc., in accordance with the particular mode of administration selected. Thus, for example, and without limitation, a typical pharmaceutical composition for parenteral injection is made up to contain 1 ml sterile buffered water, and 50 mg of BMP-2 activating agent. A typical composition for intravenous infusion could be made up to contain 250 ml of sterile Ringer's solution, and 150 mg of BMP-activating agent. Actual methods for preparing parenterally administrable compositions are known or apparent to those skilled in the art and are described in more detail in, for example, Remington's Pharmaceutical Science, 15th ed., Mack Publishing Company, Easton, Pa. (1980).

In various aspects, the pharmaceutical compositions are in the form of a sterile injectable aqueous, oleaginous suspension, dispersions or sterile powders for the extemporaneous preparation of sterile injectable solutions or dispersions, which are combinable with a controlled release formulation. The suspension is formulated according to the known art using those suitable dispersing or wetting agents and suspending agents which have been mentioned above. The sterile injectable preparation is also contemplated as a sterile injectable solution or suspension in a non-toxic parenterally-acceptable diluent or solvent, for example as a solution in 1,3-butane diol. In some embodiments, the carrier is a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity is maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms is brought about by various antibacterial and antifungal agents, for example, parabens, chlorobutanol, phenol, sorbic acid, thimerosal, and the like. In many cases, it will be desirable to include isotonic agents, for example, sugars or sodium chloride. In certain aspects, prolonged absorption of the injectable compositions is brought about by the use in the compositions of agents delaying absorption, for example, aluminum monostearate and gelatin.

Compositions useful for administration may be formulated with uptake or absorption enhancers to increase their efficacy. Such enhancers, include, for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the methods or the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action.

### Administration and Dosing

In one embodiment, administration is performed at the site of a damaged cartilage needing treatment by direct injection of the controlled release formulation into the site or via a depot or implant comprising a controlled release formulation releasing a BMP-activating agent. In some aspects, controlled release formulations are delivered to a subject in need at multiple sites. It is contemplated that the multiple administrations are rendered simultaneously or administered over a period of time. Additionally, the controlled release formulation described herein is administered as necessary to treat cartilage damage, and is administered on a period basis, for example, daily, weekly, bi-weekly, monthly, bi-monthly, every three months, every 4 months, every 6 months, or yearly.

Also contemplated in the present invention is the administration of multiple agents, such as a controlled release composition in conjunction with a second agent useful for treatment of a cartilage disorder, e.g., pain relievers or other biological molecules (e.g., cytokines or growth factors), as described below. In various embodiments, it is contemplated that these agents are given simultaneously, optionally in the same formulation or in different formulations. The second agent need not be in a controlled release formulation. It is further contemplated that the agents are administered in a separate formulation and administered concurrently, with concurrently referring to agents given within 30 minutes of each other.

In another aspect, the second agent is administered prior to administration of the controlled release formulation comprising a composition that increase BMP expression. Prior administration refers to administration of the second agent within the range of one week prior to treatment with the controlled release composition, up to 30 minutes before administration of the controlled release composition. It is further contemplated that the second agent is administered subsequent to administration of the controlled release composition. Subsequent administration is meant to describe administration from 30 minutes after controlled release composition up to one week after controlled release composition administration.

In certain embodiments, the controlled release formulation is administered such that the equivalent of about 1 ng/day, about 10 ng/day, about 100 ng/day, about 1 μg/day, about 10 μg/day, about 25 μg/day, about 50 μg/day, about 100 μg/day, about 150 μg/day, about 200 μg/day, about 500 μg/day, about 1 mg/day, about 5 mg/day, about 10 mg/day, about 20 mg/day, about 50 mg/day, about 75 mg/day, about 100 mg/day, or about 150 mg/day of a composition that increases BMP expression is delivered to the site of cartilage injury. Additionally, in some aspects, the amount of composition delivered is in a range from about 0.1 mg/kg to about 50 mg/kg. In a related embodiments, the composition is administered at about 0.1 mg/kg, about 0.25 mg/kg, about 0.5 mg/kg, about 1 mg/kg, about 2 mg/kg, about 2.5 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 40 mg/kg, about 45 mg/kg or about 50 mg/kg. Determination of an appropriate dosage will necessarily depend on the age and/or condition of the recipient, and determination of such a dosage is well within the skill level of a ordinary clinician.

### Cartilage Injury

The present invention is useful to treat cartilage degeneration in various situations, including natural degradation of cartilage over time, a degenerative disease causing cartilage breakdown or damage due to injury or trauma to the cartilage.

Exemplary disorders or disease causing cartilage damage include, but are not limited to, chronic arthridites, rheumatoid arthritis, osteoarthritis, juvenile arthritis, ankylosing spondylosis, HIV-related arthritis, psoriatic arthritis, Chondromalacia Patella, Chondrosarcoma, Costochondritis, Enchondroma, Hallux Rigidus, Osteochondritis Dissecans (OCD), Osteochondrodysplasias, Relapsing Polychondritis.

It is contemplated that the methods and compositions of the invention are useful to repair or retard cartilage damage in arthritis patients. Administration is carried as appropriate, described herein, and in one aspect, the controlled release formulation is administered via injection by fluoroscope guidance to a site of cartilage injury associated with arthritis. The method is useful in the administration of the controlled release formulation to any site of cartilage injury.

The efficacy of administration of the method and compositions to humans afflicted with arthritis is evaluated using three different criteria: the Paulus Response Criteria, the American College of Rheumatology Criteria, and the Protocol Response Criteria. According to the Protocol Response Criteria, a positive response is scored when a 30% improvement in tender and swollen joint counts is achieved in a subject.

In the American College of Rheumatology Response Criteria, a positive response is scored when: a) a 20% improvement in tender and swollen joint counts is achieved and b) there is a 20% improvement in any 3 of the following: (1) patient global score; (2) physician global score; (3) patient pain score; (4) CLINHAQ (clinical health assessment questionnaire); (5) ESR (erythrocyte sedimentation rate).

A positive result is scored in the Paulus Response Criteria when 4 of the following 6 criteria are satisfied: a) a 20% improvement in (1) tender joint score; (2) swollen joint score; (3) duration of morning stiffness (4) ESR; b) a. 40% improvement in (5) physician global score; (6) patient global score.

The protocols required subject examination by a physician of each subject participating in the study at 2, 4, 8, 12, 16, 20, and 24 weeks from the baseline date (date of entry into the study). During each examination the physician evaluates the subject for the criteria included in the Paulus Response, the American College of Rheumatology Response, and the Protocol Response evaluations.

It is further contemplated that the present methods and compositions are administered in combination with other medications already prescribed for treatment of cartilage disorders, such as analgesics, NSAIDS, steroids, anti-inflammatories, glucosamine and/or chondroitin sulfate tablets, biological molecules such as interferon alpha or beta, and other cytokines or growth factors, tumor necrosis factor inhibitors, therapeutic antibodies, such as anti-CD20 antibody, and other therapeutics known in the art.

### Kits

As an additional aspect, the invention includes kits which comprise one or more compounds or compositions useful in the method of the invention packaged in a manner which facilitates their use to practice methods. In one embodiment, such a kit includes a controlled release formulation comprising a composition (for example and without limitation, a statin or derivative thereof or other BMP activating agent) that increase BMP expression. In one embodiment, the controlled release formulation is packaged in a container such as a sealed bottle or vessel with a label affixed to the container or included in the package that describes use of the formulation comprising a compound or composition in practicing the method. In one embodiment, the compound or composition is packaged in a unit dosage form. The kit may further include a device suitable for administering the composition according to a specific route of administration. In one embodiment, the device is a syringe for injection of the hydrogel.

Additional aspects and details of the invention will be apparent from the following examples, which are intended to be illustrative rather than limiting.

### EXAMPLES

### Example 1

### Simvastatin Induces Intervertebral Disc Cell Proliferation and BMP-2 Expression

Recent experiments using bone morphogenic proteins (BMPs) to facilitate the regeneration of intervertebral disc (IVD) have shown that exogenously supplemented recombinant human BMP-2 simulates the biosynthesis of proteoglycan and collagen type II in both chondrocytes (Sailor L Z. et al., J Orthop Res, 14:937-45, 1996) and IVD cells (Kim D J. et al., Spine, 28(24): 2679-84, 2003). These molecules can be also increased in cartilaginous matrices when endogenous BMP-2 expression is up-regulated. The cholesterol-lowering drug statin that inhibits 3-hydroxy-3-methylglutaryl coenzyme A (HMG Co-A) reductase has been observed to increase expression of BMP-2 and in turn stimulate bone formation (Mundy G. et al., Science 286(5446): 1946-9, 1999) and chondrogenic phenotype (Hatano H. et al., J Orthop Sci, 8(6): 842-8, 2003). The effects of statins on (IVD) cells have not been elucidated, however. This study is aimed to investigate whether statins are anabolic to chondrogenic expression of IVD cells via up-regulating BMP-2 and evaluate the potential of statins as alternative growth factors for IVD regeneration.

### Materials and Methods

Disc cell isolation: Nucleus pulposus and inner anulus fibrosus cells of lumbar intervertebral discs from Sprague-Dawley rats were harvested by the enzymatic digestion and grown in monolayer until reached confluence.

Monolayer and Three-dimensional cell culture: Cells were trypsinized and subcultured in 12-well plate or embedded and cultured in alginate beads supplemented with complete DMEM/F12 medium after primary culture. When the cells were 80% confluent, simvastatin was added to the medium to reach the final concentration at 0.3-3 μM.

Quantitative real-time PCR: Total RNAs were extracted and cDNAs were synthesized using Invitrogen SUPERSCRIPT™ first-strand synthesis kit. Aggrecan, type II collagen and BMP-2 mRNA expression were quantified using ABI PRISM® 7700 Sequence Detection System.

Proteoglycan Production Assay: 1,9-Dimethylmethylene blue (DMMB) staining for sulfated glycosaminoglycans (sGAG) was used to measure proteoglycan production. Total sGAGs in the media or alginate beads were normalized according to counted cell number.

Statistical analysis: The data were expressed as mean±standard deviation. One-way ANOVA and Dunnett post-hoc test were used to determine the significant difference.

### Results

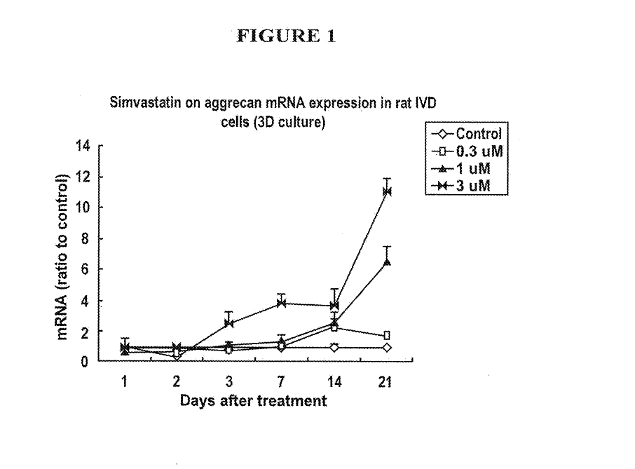

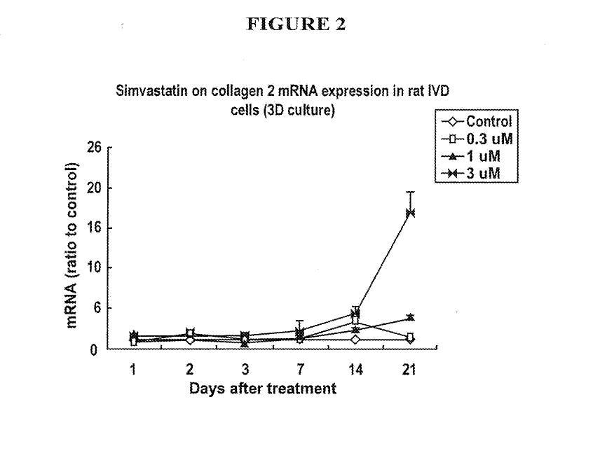

The effect of various concentration of simvastatin on IVD cell proliferation was investigated prior to the treatment to determine the range of cell tolerance. Simvastatin inhibited the IVD cells proliferation when the dose was over 3 μM. Simvastatin concentrations of 0.3-3 μM were chosen to conduct the main observations. In monolayer culture, 3 μM simvastatin began to increase BMP-2 mRNA expression at day 2 and reached maximal at day 3 by 2-fold. However, both aggrecan and collagen II mRNA expression in the treated group did not show a difference compared with the control. In 3-D culture, the data showed that simvastatin at a concentration of 3 μM continuously elevated BMP-2 mRNA expression from day 3 up to day 7, when the peak level was present (5-fold). As a consequence responsiveness to elevated BMP-2, aggrecan was expressed increasingly from day 3 to day 21 by 11-fold (FIG. 1), while collagen type II showed slight delay in the increase after day 7, the increase was sustained to day 21 by 16 fold (FIG. 2).

These results show that the susceptibility of IVD cells to stimulation of growth by simvastatin significantly differed based upon the culturing systems. The three-dimensional culture system showed greater influence on IVD cells, likely because it resembles the favorable environment for IVD cells treated with statin to retain the chondrogenic phenotype for a longer duration compared to monolayer culture of the cells.

### Example 2

### Effects of Simvastatin on Intervertebral Disc Phenotype

In order to determine the effects of the statin compounds on intervertebral disc (IVD) phenotype and expression of various proteins known to be involved in chondrocyte growth, additional studies of simvastatin on cultured IVD cells were carried out.

### Materials and Methods

IVD Cell Isolation. Lumbar IVDs from Sprague-Dawley rats (Harlan Sprague-Dawley, Indianapolis, Ind.) aged 6-8 weeks were harvested immediately after euthanasia. NP and inner AF, including the transition zone (the area between the AF and the NP) were carefully removed from the disc and placed in DMEM/F12 medium (Gibco, Grand Island, N.Y.). Using scissors, the disc tissues were minced into pieces of approximately 2 mm³ in volume, and were digested in a medium composed of DMEM/F12 containing 5% Fetal Bovine Serum (FBS) with 0.2% pronase (Sigma-Aldrich, St. Louis, Mo.) and 0.004% deoxyribonuclease II (Sigma-Aldrich) for 90 min at 37° C. under gentle agitation. The tissues were washed 3 times with Dulbecco's phosphate buffered saline (D-PBS) followed by digestion under the same conditions, except that the pronase was replaced with 0.04% collagenase type II (Sigma-Aldrich) for 6 hrs. After enzymatic digestion, the cells were washed and filtered through a 70-μm cell strainer and seeded into 75-cm² flasks at a density of 2.5×10⁴ cells/cm2 in DMEM/F-12 containing 10% FBS in a 5% CO₂ incubator for 10-14 days. Culture medium was changed on day 6 after cell seeding, and twice per week thereafter.

Cell Culture in Alginate Beads. When the primary cell culture became confluent, the cells were trypsinized and washed twice with D-PBS and re-suspended in sterile 0.15 M NaCl containing 1.2% low-viscosity alginate (Sigma-Aldrich) at a density of 1×10⁶ cells/ml. The cells were then slowly expressed through a 21-gauge needle attached to a 10-ml plastic syringe in a drop-wise fashion into 102 mmol/L CaCl₂ solutions. After 15 min, the newly formed alginate beads (containing about 12,000 cells/bead) were washed 3 times with sterile 0.15 M NaCl solution followed by 2 washes with DMEM/F-12 medium. About 45 beads were cultured in each well of a 6-well plate and were finally placed in DMEM/F12 medium with 10% FBS media+2 mM L-glutamine (Gibco)+50 μg/mL vitamin C (Sigma-Aldrich). Three days later, the medium was changed and treated with different doses of simvastatin (LKT Laboratories, St. Paul, Minn.) for 1, 2, 3, 7, 14, and 21 days. For long-term treatment (days 7, 14, and 21), the medium was changed twice every week and simvastatin was added with medium change each time during the experiment. To remove cells from the alginate bead, wells were rinsed 3 times with 0.15 M NaCl with gentle pipetting into the well. The rinse solution was incubated for 1 min and was aspirated off. Dissolving buffer (55 mmol/L sodium citrate and 0.15 M NaCl, pH 6.0) 3 times the volume of alginate was added to the wells, and plates were incubated at 37° C. for 15 min with gentle shaking. Cells were pelleted by centrifugation and the dissolved solution was collected for sGAG content measurement. The cell pellet was washed with D-PBS once and collected for DNA content determination or RNA extraction. All experiments were performed in triplicate.

sGAG Assay. The sGAG content of the dissolved solution at each time point tested was assayed using the DMMB method (45, 46). The sample solution of the dissolved solution (20 μL) was mixed gently with 200 μL DMMB dye solution in a 96-well culture plate, and the optical density was checked immediately at 525 nm wavelength filter. A standard curve was constructed using serial dilutions of chondroitin sulfate (Sigma-Aldrich). Total sGAG in the media were normalized by DNA content and presented as a ratio to the untreated control.

DNA Assay. The DNA content was measured using the Hoechst dye 33258 method. Briefly, the cell pellets from alginate beads were exposed to papain solution containing 10 U/mL papain (Sigma-Aldrich), 5 mM cystein-HCl and 5 mM EDTA and were incubated at 60° C. for 24 h. Papain digest (100 μl) was mixed with 1 ml of Hoechst dye 33258 solution (Sigma-Aldrich) in a cuvet, and the emission and excitation spectrums were measured by luminescence spectrometer with TBS 380 at 460 nm and 360 nm, respectively. Standard curves were generated at the time of each measurement using known concentrations of calf thymus DNA (Sigma-Aldrich).

RNA Isolation and Reverse Transcription. Total RNA was extracted using the Trizol (Invitrogen, Carlsbad, Calif.) reagent followed by RNeasy Mini Kit (Qiagen, Inc., Valencia, Calif.) and DNase digestion with the RNase-free DNase set (Qiagen). Concentration of total RNA was determined at 260 nm with a spectrophotometer. Reverse transcription was carried out at 42° C. for 50 min in 20-μL volume with 100 ng of total RNA using the SuperScript First-Strand Synthesis System (Invitrogen, Carlsbad, Calif.).

Quantitative Real-Time Polymerase Chain Reaction. Real-time polymerase chain reaction (PCR) was used to quantify gene expression levels of type II collagen, aggrecan, and BMP-2. GAPDH was used as an internal control and the TAQMAN® Real-Time PCR Kit (Applied Biosystems, Foster City, Calif.) was implemented. Reaction volume of 30 μl included 1 μL of cDNA, 1.5 ul of 20× of each primer/probe, and 12.5 μL of TAQMAN® PCR Master Mix (2×) (Applied Biosystems). Real-time PCR was performed using the following 3-step protocol: Step 1, 50° C. for 2 min; Step 2, 95° C. for 10 min; and Step 3, (95° C. for 15 sec, 60° C. for 1 min) times 45 cycles using the Gene Amp 7700 Sequence Detection System (Applied Biosystems). A positive standard curve for each primer was obtained by real-time PCR with serially-diluted cDNA sample mixture. The reaction efficacy of each gene was determined using the standard curves. The quantity of gene expression of aggrecan, type II collagen, and BMP-2 were calculated with standard samples and normalized with GAPDH internal control. The amount of mRNA expression in each group receiving the simvastatin treatment was compared with that demonstrated in the non-treatment control group and reported as a ratio.

BMP Inhibition Experiment. Noggin is the BMP ligand antagonist that binds to BMP-2 in a highly-specific manner and prevents these BMPs from activating their cognate receptors. A form of mouse noggin (noggin-FC, Sigma-Aldrich) was used in the experiments to determine the effect of specifically blocking BMPs after simvastatin treatment in 3-dimensional alginate culture. Noggin at different concentrations (500 and 3000 ng/mL) was applied to cells in the absence or presence of simvastatin (3 μM). On day 7 and/or day 14, the cells were removed from the alginate beads and the gene expression of type II collagen and aggrecan was determined.

Mevalonate Experiment. To determine if the effect of simvastatin on gene expression is through mevalonate pathway, 200 μM mevalonate (Sigma-Aldrich) was applied to cells culturing in the 3-dimensional alginate beads in the absence or presence of simvastatin (3 μM). On days 7 and 14, the cells were removed from the alginate beads and the gene expression of type II collagen, aggrecan, and BMP-2 were determined.

MTT assay. Cell proliferation was determined by a modified MTT assay (Sigma-Aldrich) (47, 48). Briefly, at each time point, the medium was aspirated and washed with PBS once. MTT solution (0.5 mg/ml) in PBS was added and incubated at 37° C. for 1.5 hr. The cells were removed from the alginate bead and pelleted by centrifugation. The cell pellet was then washed with D-PBS once and 10% Triton X-100, 0.1 N HCl in 2-propanol solution was added to each well to dissolve the formazan at 4° C. for 3 hrs. The dissolved solution was centrifuged and the absorbance was determined immediately using a spectrophotometer at 540 nm wavelength filter. All experiments were performed in triplicate.

Statistical Analysis. DNA content, sGAG content, and mRNA results for the control group were normalized as 1 at each time point in the background level. Data for the simvastatin treated groups were expressed as a ratio to control. One-way ANOVA with Dunnet post-hoc analysis was used to determine the difference between each group at the same time point. P value of <0.05 was considered statistically significant.

### Results

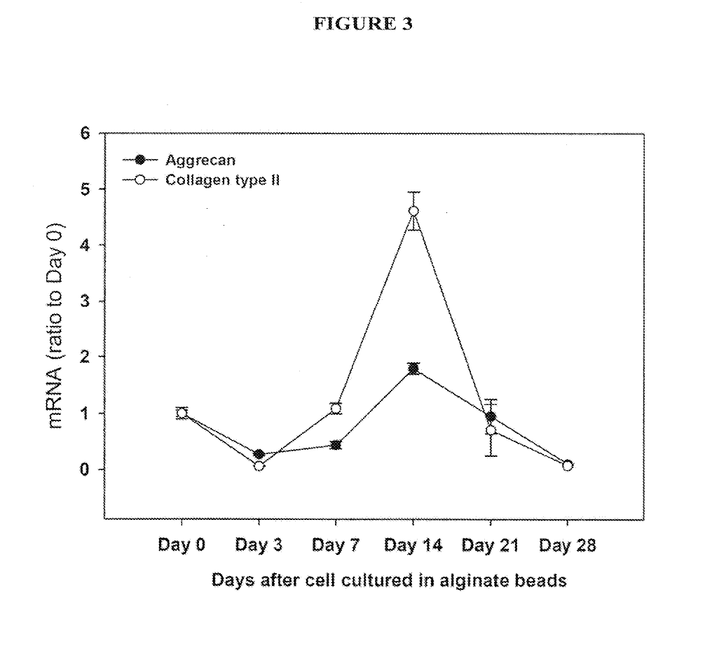

Effects of Culture Time on Aggrecan and Type II Collagen mRNA Expression in IVD Cells in Alginate Beads. To determine the effective duration for treatment with simvastatin, the change in chondrogenic genotype expression of IVD cells in an alginate bead culture system along a time course was investigated. After IVD cells were embedded in alginate beads, the mRNA expression of both aggrecan and type II collagen decreased at day 3 and was recovered to the initial level (day 0) at day 7. Gene expression of aggrecan and type II collagen reached their maximal levels at day 14. When cultured in alginate beads, IVD cells can keep their chondrogenic phenotype as long as 21 days (FIG. 3). Therefore, 21 days was chosen as the time span for treatment in future studies.

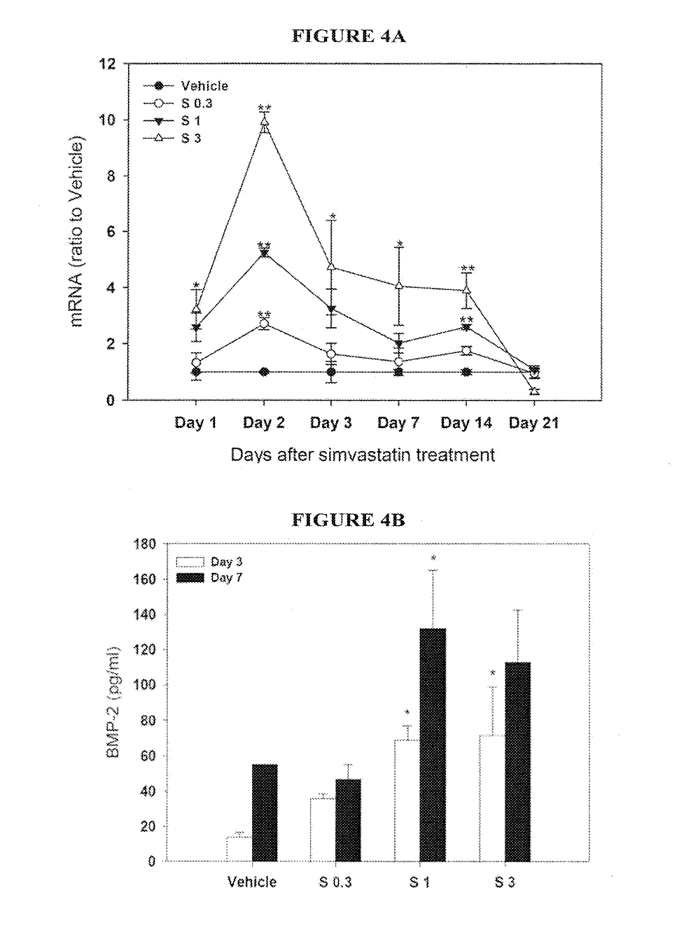

Simvastatin Upregulated BMP-2 mRNA Expression in IVD Cells. The time course for the effect of simvastatin on BMP-2 mRNA expression was first examined in rat IVD cells. FIG. 4 shows that simvastatin increased BMP-2 mRNA expression (FIG. 4A) in a dose-dependent manner. Simvastatin in 0.3 μM and 1 μM doses significantly increased BMP-2 mRNA expression at day 2. At the dose of 3 μM, BMP-2 mRNA was significantly upregulated at day 1, reaching its maximum at day 2, and still increased significantly on days 3-14 when compared to the control group at each time point. The protein levels of BMP-2 were also consequently increased with 1 and 3 μM simvastatin treatment at 3 and 7 days (FIG. 4B). There was no significant difference at day 21 in the simvastatin-treated groups compared with the control group.

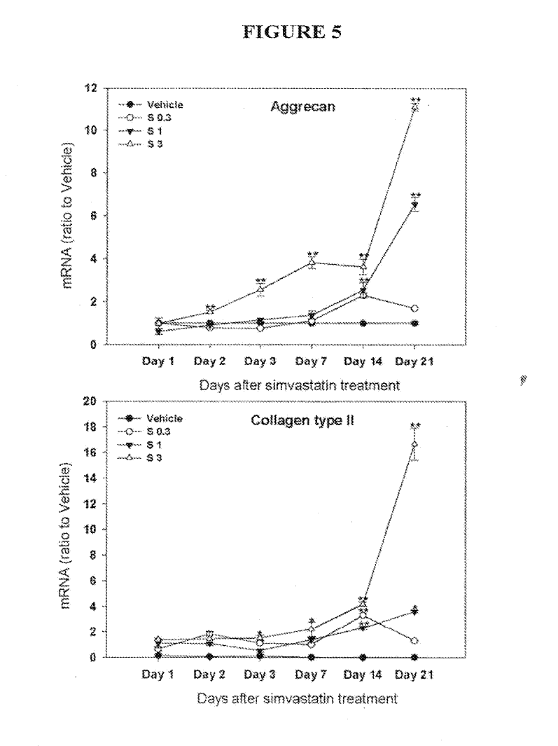

Simvastatin Increased Aggrecan and Type II Collagen mRNA Expression in IVD Cells. Next, the effect of simvastatin on chondrocytic marker gene expression in IVD cells cultured in alginate beads was tested. At 3 μM, simvastatin significantly upregulated aggrecan mRNA expression at day 2 (FIG. 5A), and it reached the maximum at day 21. Similarly, simvastatin also increased type II collagen mRNA expression starting from day 3, and the maximal level (16-fold increase) was obtained at day 21 (FIG. 5B).

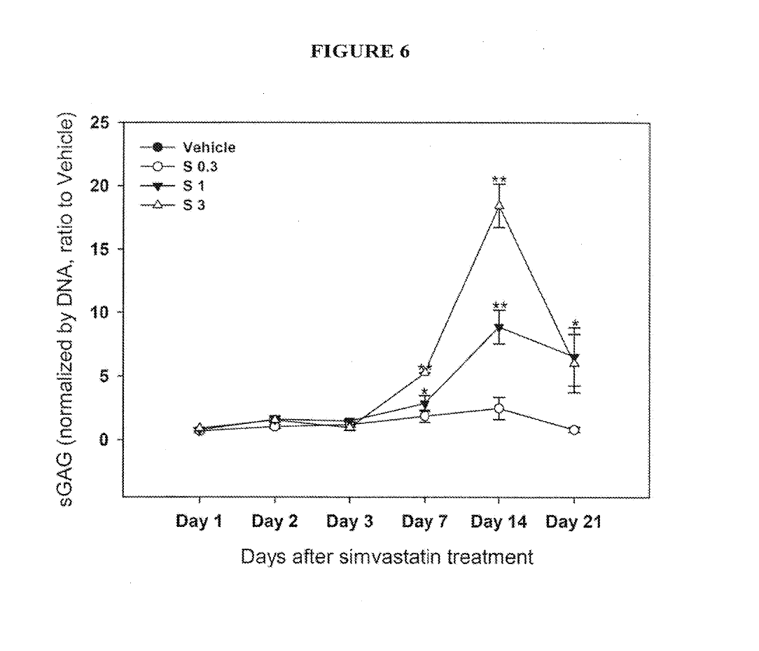

Simvastatin Increased Sulfated Glycosaminoglycan (sGAG) Production in IVD Cells. FIG. 6 illustrates that simvastatin increased sGAG content significantly with doses of 1 and 3 μM at 7, 14, and 21 days. The induced sGAG reached its peak level after 14 days of treatment with simvastatin. At day 21, despite a drop in the increased amount, groups treated with 1 and 3 μM simvastatin still maintained a significant increase in sGAG content compared to the control group.

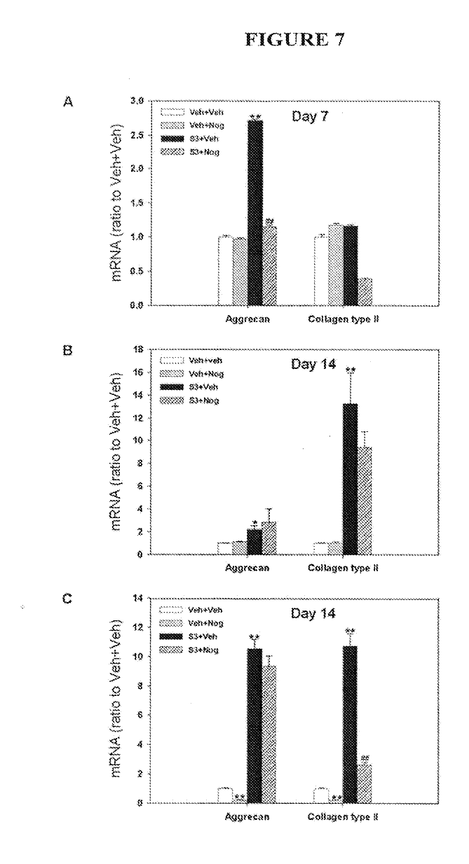

Effect of Noggin on Gene Expression Induced by Simvastatin in IVD Cells. Noggin alone at 500 ng/ml had no interference on aggrecan and type II collagen mRNA expression (FIG. 7A). However, it significantly blocked both upregulated gene expression induced by 3 μM of simvastatin at day 7, but this suppression did not endure up to day 14 (FIG. 7B). When the dose of noggin was increased to 3 μg/ml, it inhibited both gene expressions significantly compared with the Veh+Veh group. Moreover, noggin at this dose significantly attenuated the upregulated type II collagen gene expression on day 14, whereas it failed to block the upregulated aggrecan gene expression induced by simvastatin (FIG. 7C).

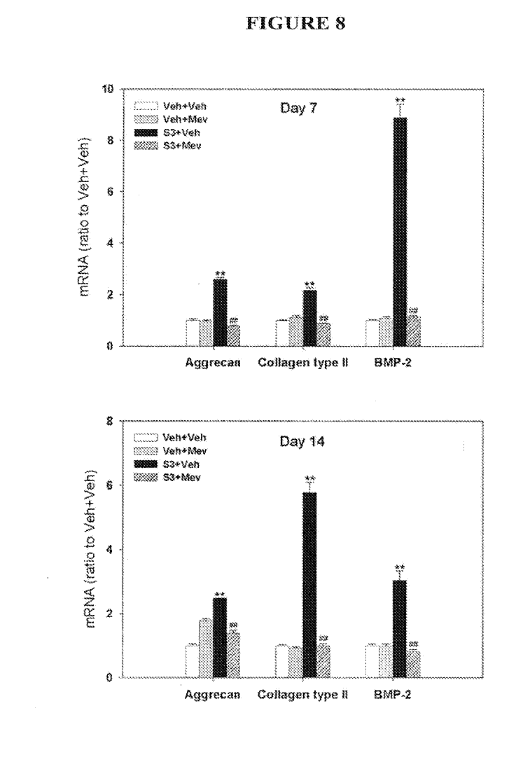

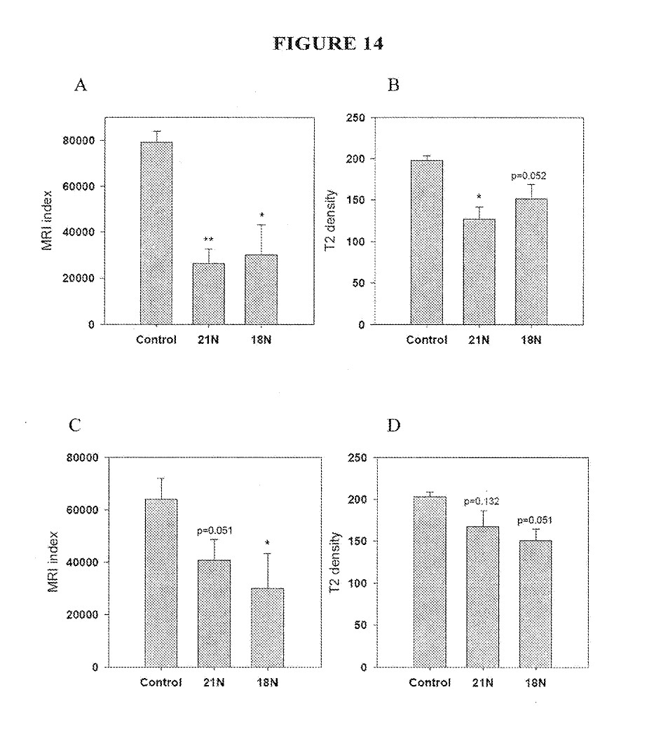

Effect of Mevalonate on Gene Expression Induced by Simvastatin in IVD Cells. Since the direct metabolite of HMG-CoA reductase, mevalonate, initiates the cascade of cholesterol synthesis and is responsible for the production of isoprenoid intermediates, the ability of simvastatin suppression of mevalonate to dominate BMP-2-mediated regulation was analyzed. Mevalonate alone at 200 μM had no effect on aggrecan, type II collagen, or BMP-2 gene expression in IVD cells cultured in 3-dimensional alginate beads. Consistently, simvastatin at the dosage of 3 μM in this set of experiments significantly increased BMP-2, aggrecan, and collagen type II gene expression. When pretreated with mevalonate, the upregulated gene expression by simvastatin was completely reversed by days 7 (FIG. 8A) and 14 (FIG. 8B). The results indicated that with the presence of mevalonate in the culture treated with simvastatin, upregulated BMP-2 expression was completely reversed at all time points tested, as were the induced aggrecan and type II collagen.

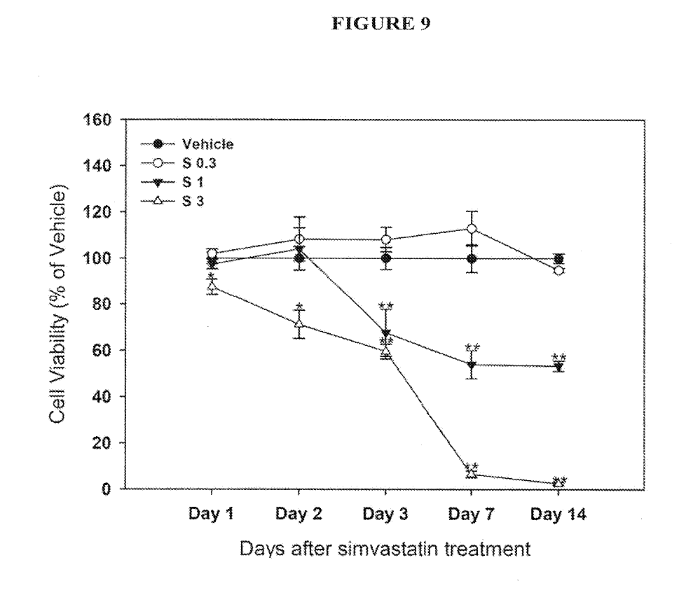

Effect of Simvastatin on IVD Cell Viability. FIG. 9 illustrates that simvastatin significantly decreased IVD cell viability in a dose- and time-dependent fashion. Simvastatin at the dose of 0.3 μM had no effect on cell viability at the time points tested. However, a significant decrease in cell viability was observed from day 3 and thereafter when the cells were treated with 1 μM of simvastatin. Cell viability decreased at all time points tested when cells were treated with 3 μM of simvastatin.

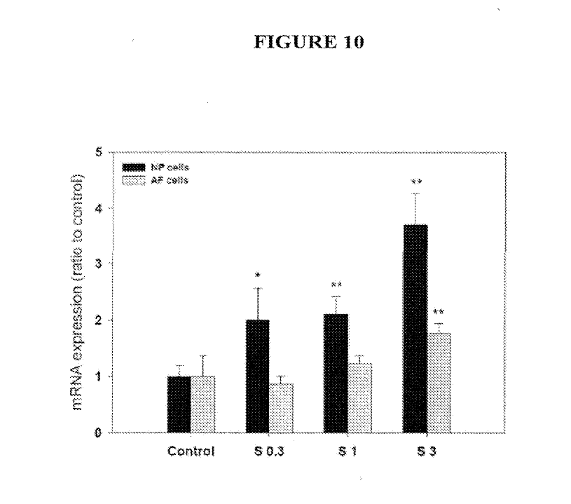

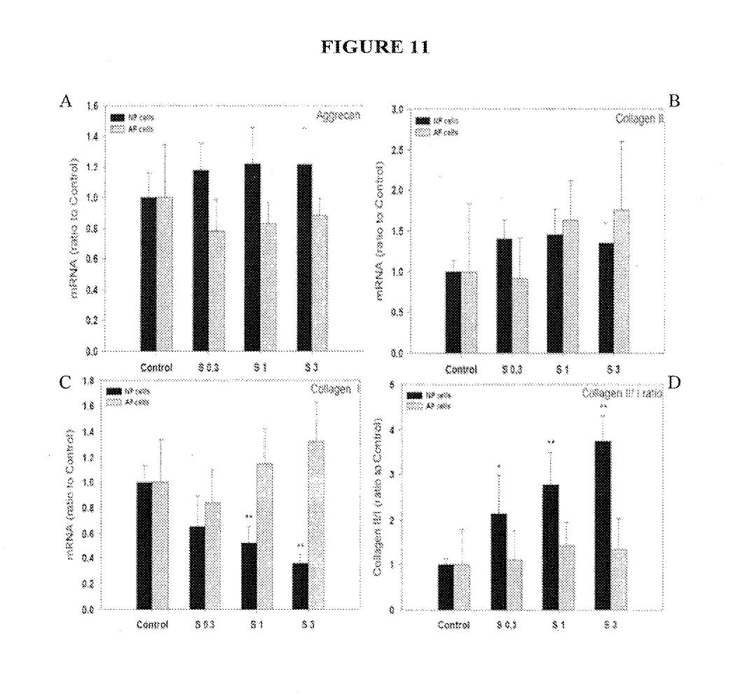

Effect of Simvastatin on Chondrocyte Phenotype and Protein Expression: Simvastatin Stimulates Chondrogenic Phenotype of Degenerative Human Intervertebral Disc Cells. When IVD cells from human patients with degenerative disc disease (DDD) were exposed to simvastatin, these cells were stimulated to maintain or even increase the chondrogenic phenotype in a dose-dependent manner. However, there were differences in the expression pattern from that in rat IVD cells discussed above. Simvastatin up-regulated BMP-2 mRNA expression in both of the human NP and AF cells as observed in rat cells (FIG. 10). In addition, histological examination showed that both the NP and AF cells expressed the BMP-2 receptor, BMPRII, indicating that both cell types are susceptible to the upregulation of BMP-2 induced by simvastatin to mediate the determined pathways as determined by histology. However, the mRNA expressions of aggrecan and type II collagen were not affected when the human NP cells were treated with simvastatin at the same doses (0.3 to 3 μM) used to in rat cells. But simvastatin did suppress type I collagen mRNA expression in a dose dependent manner, and therefore significantly increased the ratio of type II to type I collagen (FIG. 11). This phenomenon was only observed in the human NP cells. Simvastatin did not change the mRNA expression of aggrecan, collagen type II and collagen type I in the human AF cells.

It has been shown that type II collagen is one of the featured markers of differentiated chondrocytes in cartilage and NP, whereas type I collagen is mainly synthesized by dedifferentiated chondrocytes. The ratio of mRNA levels of Col II/Col I was therefore defined as “differentiation index” to evaluate the capacity of chondrogenesis in chondrocytes (Yang, Saris et al. Osteoarthritis & Cartilage 14(6): 561-70, 2006; Yang, Saris et al. Tissue Engineering 12(10): 2957-2964, 2006). The fact that simvastatin increased the ratio of Col II/Col I in human disc cells suggests that simvastatin may have restrained the dedifferentiation of the human NP cells (particularly for chondrocyte-like cells in NP) in the degenerated discs, which would have assisted the maintenance of their chondrogenic phenotype.

### Discussion

The studies above demonstrated that simvastatin increased BMP-2 mRNA expression in rat IVD cells in vitro when cultured in 3-dimensional alginate beads. In addition, aggrecan and type II collagen gene expression as well as proteoglycan product were upregulated, demonstrating the anabolic effect of simvastatin on IVD cells. More importantly, these results provided the first evidence that increased mRNA expression of aggrecan and type II collagen induced by simvastatin is partially mediated by upregulated BMP-2 through mevalonate pathway.

In this study, the majority of the cells collected from NP and inner AF were mainly referred to as chondrocyte-like cells, which primarily synthesize aggrecan and type II collagen (20-23). To investigate the genotypic change of the cells and determine the effective duration of simvastatin treatment, IVD cells were first cultured in a monolayer and also in alginate beads resembling a 3-dimensional environment, followed by monitoring the mRNA change of aggrecan and type II collagen. The data showed that in the monolayer culture, IVD cells lost their basal level of chondrogenic expression very quickly, and the gene expression of aggrecan and type II collagen became undetectable after day 7. However, when the cells were cultured in alginate beads at a density of 1×10⁶ cells/ml, the mRNA expression of aggrecan and type II collagen was sustained for 21 days. This result was consistent with previous studies stating that 3-dimensional culture in alginate beads provides a more favorable environment for IVD cells to maintain chondrogenic phenotype for longer duration compared to a monolayer culture (24, 25). Based on these results, the 3-dimensional culture system with the time course of 21 days of simvastatin treatment was chosen for additional study.

Earlier studies have shown that statins upregulated mRNA expression of BMP-2 in various cell types such as osteosarcoma cells, bone marrow stromal cells, osteoblasts, chondrocytes, and human vascular smooth muscle cells (16-19). Similar to these findings, the present data demonstrated that simvastatin also increased mRNA expression of BMP-2 in IVD cells in a dose-dependent manner. Moreover, the upregulated response of BMP-2 was present as quickly as 1 day after treatment and later reached the maximal level on day 2. Elevated BMP-2 gene expression persisted up to 14 days compared to the basal level, yet in a decreasing manner. This may imply that a negative feedback mechanism may exist to regulate BMP-2 expression, which requires further investigation. This is the first observation of the long-term profile of simvastatin on BMP-2 gene regulation.

Given the anabolic effect of BMP-2 on IVD cells, whether simvastatin-increased endogenous BMP-2 could stimulate expression of the chondrogenic phenotype of IVD cells was investigated. The results presented herein further demonstrated that simvastatin increased aggrecan and type II collagen mRNA expression as well as sGAG content up to 21 days, indicating the anabolic action of simvastatin on rat IVD cells. Since the hallmark of disc degeneration is the loss of proteoglycan, the ability of simvastatin to promote the anabolism of IVD cells over a long period supports the hypothesis that small molecules such as statins are a potential treatment for disc degeneration. Although the peak level of sGAG presented at day 14, the aggrecan mRNA level was still highly expressed until day 21 compared to the control group, further illustrating the persistent induction of simvastatin. Comparing the consequences of these time factors, the data showed that upregulation of BMP-2 induced by simvastatin occurred prior to the elevated expression of aggrecan and type II collagen, raising the possibility that the stimulation of aggrecan or type II collagen by simvastatin was through the upregulated BMP-2. To identify the role of BMP-2 in the upregulation of aggrecan and type II collagen mRNA expression induced by simvastatin, the noggin antagonist study was conducted.

Noggin binds to BMP-2 with high affinity and prevents BMP-2 from binding to its receptor, thus blocking the downstream effect of BMP-2 (26-28). The common dose range of noggin (500 ng/ml), which blocks the BMP-2-induced effect in various assays (29-32), was chosed for the study. The results showed that pretreatment with noggin completely blocked the upregulation of aggrecan and type II collagen mRNA expression induced by simvastatin at day 7, but not at day 14. Next, considering that the 3-dimensional alginate beads used to culture cells may form a barrier that prevents noggin from reaching the entrapped cells, the noggin dose was increased to 3 μg/ml, which blocked BMP-2 activity over 90% (33). The current data illustrated that the high dose of noggin alone could significantly inhibit the basal aggrecan and type II collagen mRNA expression at a longer period (day 14), indicating the necessity of BMPs in regulating the chondrogenic genotype of IVD cells. Surprisingly, the result also indicated that noggin at this dose only blocked the upregulation of type II collagen, but not aggrecan mRNA expression induced by simvastatin on day 14. These findings imply that the increased expression of collagen type II by simvastatin is more dependently-mediated by BMP-2, whereas aggrecan seems less dependent on this influence. However, since BMP-2 contains the heparin-binding sites, the ionic interactions between the basic residues in the heparin-binding site of BMP-2 and carboxylate groups of alginic acid may exist, resulting in a relative high concentration of BMP-2 surrounding the cells in the beads due to restricted diffusion (34, 35). If this is true, the possibility exists that the expression of aggrecan in IVD cells may be more sensitive (low activation threshold) than that of type II collagen in response to the undisturbed BMP-2 accumulated in alginate beads. Previous studies also support this viewpoint by demonstrating that BMP-2 is 1.8 to 2.5 times more potent in the upregulation of aggrecan expression than that of type II collagen (7, 10). Moreover, it should also be noted that the focus of the current study was primarily on the effect of simvastatin on IVD cells and whether its action is through upregulated BMP-2. It would not be surprising if there were other BMPs excluded from the current study, such as BMP-7, that could have participated in the scheme of the induction of simvastatin, because noggin has low affinity to BMP-7 (7, 36). By modulating the upstream movement of the cholesterol synthesis pathway, statins decrease the concentrations of many important compounds including mevalonate (the direct metabolite of HMG CoA reductase) and mevalonate-derived isoprenoids. Numerous studies have demonstrated that the pleiotropic effect of statins relies on the reduction of the cellular levels of these compounds.

Mevalonate is the precursor for sterol and non-sterol isoprenoids, which are involved in membrane biogenesis, DNA replication, and protein glycosylation (37). Additionally, the mevalonate-derived isoprenoids, such as farnesyl pyrophosphate (FPP) and geranylgeranyl pyrophosphate (GGPP), are also involved in posttranslational modifications of several small G proteins, which play critical roles in intracellular trafficking and subsequent cell-signaling pathways (38, 39). The present study indicates that with the presence of mevalonate in the culture treated with simvastatin, upregulated BMP-2 expression was completely reversed at all time points tested, as were the induced aggrecan and type II collagen. Thus, it is reasonable to conclude that the induction of simvastatin on chondrogenic expression of IVD cells results from blockage of the mevalonate pathway.

However, in this study, the treatment of simvastatin also significantly decreased cell viability, especially when a high dose (3 μM) was given, even though it was the most effective dose among those tested. This observation is consistent with previous studies indicating that statins decreased cell viability in a variety of cell types, such as rheumatoid synovial cells, pericytes, smooth muscle cells, cardiac myocytes, and several types of cancer cells in vitro (40). The cause of the decreased cell viability may be attributed to the interference of FPP and GGPP, which are also involved in the control of cell adhesion, growth, and survival through protein prenylation. Blocking mevalonate pathway by statins will inhibit the prenylation of proteins such as RhoA and Ras through FPP and GGPP, resulting in alteration of cell growth and survival functions (41-44). The data presented here support use of statins to facilitate chondrogenesis in degenerative discs and other cartilage-containing areas. However, when an effective dose may also cause considerable cell damage leading to cell death, the value of the off-label benefits of statins may be reduced with an optimal range of dosage as well as potential co-treatment with other compounds.

Additionally, culture of human degenerative discs in the presence of simvastatin demonstrated that simvastatin is a potent activator of BMP-2 expression in human disc cells and helps prevent the dedifferentiation of the human NP cells, and allows maintenance of the chondrocyte phenotype of these cells. This study suggests that statins, derivatives thereof and other BMP agonists are effective at inducing a chondrocyte phenotype in damaged human disc cells.

In summary, this study demonstrates that simvastatin stimulates the chondrogenic phenotype of IVD cells partially mediated by upregulated BMP-2 through mevalonate pathway. The presented data suggest that using small molecules such as statins may shed light on developing an alternative strategy to prevent or retard disc degeneration.

### Example 3

### Methods of Inducing Disc Injury in Animals

A stab incision at the IVD to induce degeneration has become a widely used in vivo approach to investigate experimental IVD lesions. It has been conducted with various animals including rabbit (Lipson and Muir, Spine 6(3):194-210, 1981; Takaishi et al., J Orthop Res 15(4): 528-538, 1997; Nomura et al., Clin Orthop 389: 94-101, 2001), dog (Hampton et al., Spine 14(4): 398-401, 1989), sheep (Osti et al. Spine 15(8): 762-767, 1990; Moore et al., Spine 21(8): 936-940, 1996), goat (Ethier et al., Spine 19(18): 2071-2076, 1994), and pig (Kaapa et al., J Orthop Res 12(1): 93-102, 1994; Kanerva et al., Spine 22(23): 2711-2715, 1997). Stab-induced degeneration can create morphological features similar to many of those in human age-related DDD (Boos et al., Spine 27(23): 2631-2644, 2002). However, rodent models have almost never been utilized, except one experiment was undertaken by stabbing discs of rats using number 11 blade (Rousseau et al. Spine 32(1): 17-24, 2007]. Currently there is no consensus regarding the “best” model for disc degeneration, a cost-efficient, largely replicable model with consistent onset of the disorder and reproducible outcomes would be extremely valuable and attractive for searching disc regeneration regimens.

In order to study the effects of statins on disc regeneration, an animal model was developed. The needle stab injury model represents the degenerative disc disease (DDD) population that has encountered an tear in the annulus fibrosus, which eventually leads to disc degeneration. This model has been widely adopted and approved to be effective to induce significant disc degeneration. Three-month-old female Sprague-Dawley rats were used in this study. The species typically reaches skeletal maturity before 3 months of ago, so animal growth will not affect intervertebral disc (IVD) remodeling during the study.

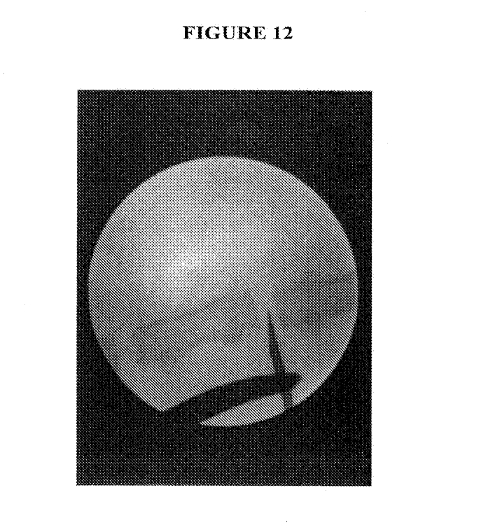

General anesthesia was administered according to the approved protocol. Briefly, the approached caudal segments were located by palpation and a small skin cross-incision (about 2 mm in length) were made to guide the needle insertion. Co6/Co7 remained undisturbed as the control level. Fluoroscopy was utilized to visualize the needle penetration and ensure the consistent depth of the stab (5 mm into the disc to depressurize the nucleus). The needle was then inserted at the center of disc level through the AF into the NP of Co5/Co6 and Co7/Co8, rotated 180°, and held for 5 seconds, as shown in FIG. 12. Two needle sizes were selected, #18(OD:1.270 mm) and #21(OD:0.813 mm) to investigate whether the size of the injury wound would affect the degeneration rate.

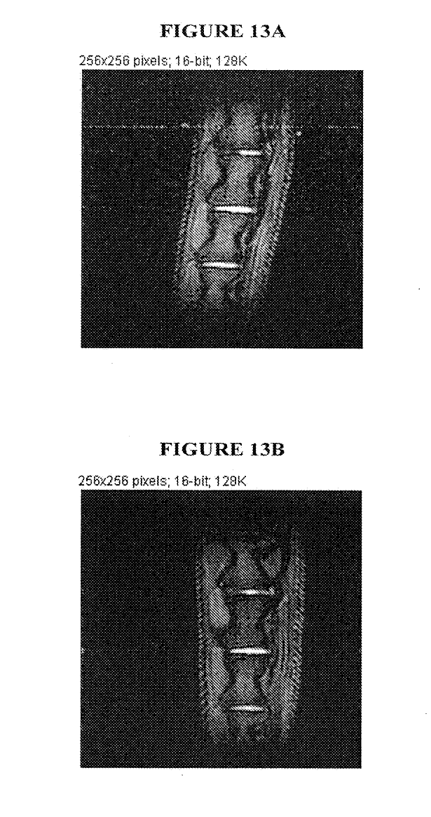

Magnetic resonance imaging (MRI) was performed on all animals at the predetermined time points of 4, 8, 12, and 24 weeks after the needle stab to monitor the progression of the disc degeneration induced by the injury. At 4 weeks, the stabbed discs started to show decreased signal intensities compared to the normal discs (control) (FIG. 13A). It was noted that the intensity of #18-stabbed discs (larger injury) was greater than that of #21-stabbed, which may be due to the stronger hypertrophic response during wound healing. At 8 weeks, the intensities of all perturbed discs were more decreased and the decreased intensities were dependent on the injury size that #18-stabbed discs presented greater dark areas than #21-stabbed discs and the normal discs as well (FIG. 13B). FIG. 14 shows the changes of the T2-weighted image intensity and the MRI index, which is defined as the product of the pixels and the corresponding image densities, along the experimental time course.

Histological evaluations of the perturbed discs shows the normal disc has a boated-looking NP which occupies at lease one half of the disc area in the midsagittal sections. The annular fibrosus is intact and the border between annulus fibrosus and nucleus pulposus is well-defined. After the injury, the disc progressively developed signs of degeneration with decreases in the NP area, disorganized AF and/or the diminishing boundary between the NP and the AF. The histological categorization was applied to grade the degeneration (4 points: normal, Grade 1; 5-7 points: Grade 2; 8-10 points: Grade 3; 11-12 points: Grade 4). By day 3 th average grade was just above 2, at day 10 the average grade was approximately 2.5, by day 17 the degeneration grade was above 3, and at 6 weeks after injury the average disc degeneration grade was above 3.5.

To test whether simvastatin loaded in a thermosensitive, biodegradable polymer injected into the perturbed disc and retard or even reverse disc degeneration, an additional needle stab model was created by stabbing the discs at Co5/Co6 and Co7/Co8 (Co6/Co7 remained intact as the control) by using #21 needles. Six weeks after the injury, a thermosensitive, biodegradable polymer was used to mix with simvastatin at 4° C., at which the polymer was in its liquid phase with a viscosity close to water. The drug-loaded polymer (2 μl of 5 mg/ml) was immediately injected into the perturbed discs, where Co5/Co6 was injected with simvastatin-loaded polymer and Co7/Co8 was injected with the polymer only. The polymer became cured (turned to gel phase) when delivered to the in vivo environment where the temperature equilibrates to the animal body temperature. MRI examinations were performed on all animals at the predetermined time points of 2 and 6 weeks after the injection to observe the signal intensity of the discs.

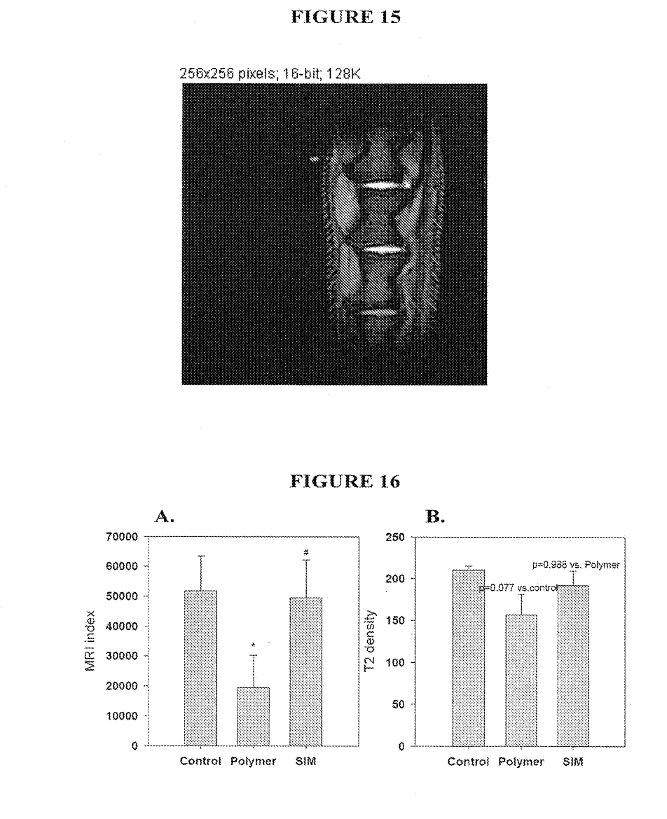

Two weeks after the injection, all perturbed discs that were injected with simvastatin-loaded polymer were fully recovered with the normal image intensity level, whereas the stabbed discs that were injected with the polymer only still presented decreased intensity as seen in the sham study. FIG. 15 shows the T2-weighted MR images of three levels in the animal at 2 weeks after the injection. FIG. 16 shows that the injection of simvastatin-loaded polymer reversed the degenerative discs back to normal intensity level and normal MRI index.

Histologically, hematoxylin and eosin (H&E) stained specimens revealed that nucleus pulposus of the intact control disc was round and had a well-defined border with annulus fibrosus. Two weeks after the injection with polymer only in the stabbed disc, the nucleus pulposus became irregular and the border between AF and NP was less obvious than that in intact control discs. The cell number in the NP of this group seemed less than that in the intact controls.

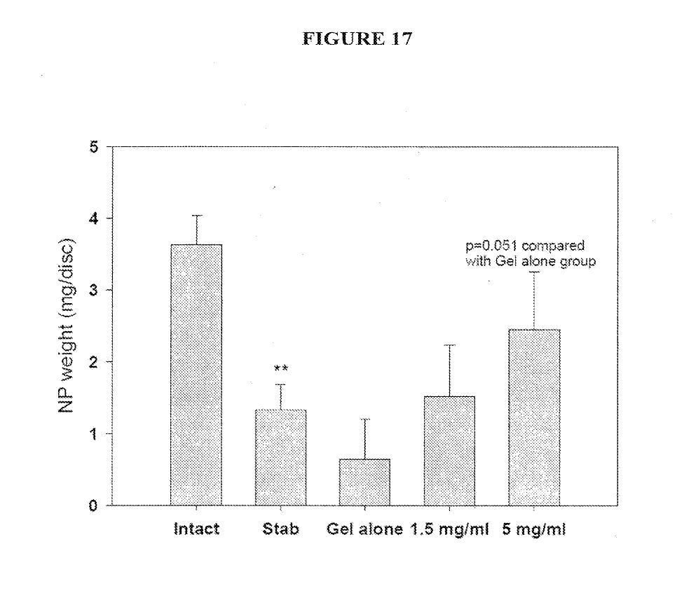

However, for the group treated with the simvastatin-loaded polymer, the NP size returned to normal although the shape of NP was still irregular. On the other hand, Safranin O, which stains proteoglycan, specifically demonstrated that simvastatin treatment significantly increased the proteoglycan content compared to that in discs injected with polymer only, implying a pertinent recovery of physiological functions of the disc. The loss of water content and sGAG are also major signs to indicate disc degeneration, as eventually the loss of both components of NP will lead to a shrunk and desiccated disc. This study also showed that the wet weight of NP was correlated to the T2 density. The T2 density of the stabbed discs was significantly decreased with a significantly lower wet weight of NP compared to that of the normal discs, whereas the wet weight of NP in the treated discs was retained after the injection of simvastatin loaded in the polymer (FIG. 17).

These results demonstrate that administration of simvastatin in a controlled release formulation directly into an injured IVD promotes repair of the disc injury. Simvastatin reduces water loss and proteoglycan loss in the disc which leads to reduced volume in the disc and further degeneration. These studies show that statins and other BMP-2 activating agents promote disc regeneration or limit disc damage and suggest these compounds are effective in treating degenerative cartilage in human patients.

### Example 4

### Porcine Model for IVD Degeneration

Although a rabbit model for IVD degeneration has been used in the development of new therapeutic interventions (Zhan et al., Journal of Huazhong University of Science and Technology Medical Sciences 24(6): 599-601; An et al., Spine 30(1): 25-31, 2005; Sakai et al., Spine 30(21): 2379-87, 2005; Masuda et al., Spine 31(7): 742-754, 2006) it is commonly held that the disc size of rabbits or rats are small, making it difficult to track the progression and identify the regional alterations in the degenerative disc with follow-up imaging. Larger animal models are desired to reproduce disc degeneration that is more relevant to man.

A study in a Sinclair minipig indicated that a stab incision led to an increase in both of interleukin-1 (IL-1) and -8 (IL-8), which are all hyperalgesic and are capable of sensitizing nociceptors (pain receptors) in tissues, and are thought to be associated with radicular pain and discogenic back pain, respectively (O'Neill et al., Spine Journal 4(1): 88-98, 2004). This finding showed that the stab-induced degeneration in pig was similar to the pathophysiological factors that may be responsible for the symptoms of human disc disease. A recent report by Yoon et al. (Journal of Neurosurgery Spine 8(5):450-7, 2008) described that limited stab injury at the AF punctured by a 3.2-mm-diameter trephine without irritating the NP allowed the degeneration progression along a natural pathway. At just 5 weeks post-injury, the lesioned discs developed Grade III disc degeneration categorized by Pfirmann imaging scales (Pfirrmann et al., Spine 26(17): 1873-1878, 2001]. Additionally, the minipig is susceptible to BMPs like rodents and humans (Zhang, et al. Spine 30(5): 512-518, 2005). Therefore, a stab injury model of IVD degeneration was developed using the Yucatan minipig. The Yucatan minipig is an accepted approximation for the human spine based on anatomical comparison of vertebra between the two species (McLain et al., Spine 27(8): E200-6, 2002).

Generally, the model requires creation of a stab injury at lumbar intervertebral disc on a porcine model (Yucatan minipig), observation of the inflammatory responses and the progression of disc degeneration in the animal and administration of the statin-loaded gel (given doses are based on the NP volume ratio of the species) into the perturbed discs that develop into Grade II-III degeneration.

First, skeletally mature (typically 6-9 months old) Yucatan minipigs are induced with 3 mg/kg Telazol (Tiletamine/Zolazepam; im), and then endotracheally intubated and maintained on continuous inhalation anesthesia with isoflurane and oxygen to effect. The operative field is kept in a strict sterile condition. Antibiotic prophylaxis, Amoxicillin 50 mg/kg is given intravenously prior to the surgery. After standard general anesthesia is carried out, the minipig is positioned prone on the operative table. By using a guidewire, the L1-2, L2-3, L3-4, L4-5, and L5-6 disc space is accessed using a fluoroscope to help guide the wire. For example, a 22 gauge spinal needle is fluoroscopically guided into the disc from a right posterolateral, extrapedicular approach entering the disc through an access triangle delineated medio-dorsally by the superior articular process, supero-ventrally by the exiting nerve root and caudally by the body of the superior endplate of the inferior vertebra. A small amount of radio-opaque tracer is injected through the needle and into the disc to assure correct placement and integrity of the disc. At L1-2, L3-4, and L5-6, a small incision is made around 7-8 cm from the midline, and the needle directed to the anterolateral portion of the disc under fluoroscopic guidance, followed by an insertion of a dilator over the guidewire. A guide cannula is then inserted over the dilator, and a trephine (3.2 mm) is used to create 2-3 mm incisions in the annulus fibrosis on the left dorsal side of the animal. The accessed L2-3 and L4-5 remain intact without injury as a control level. In previous animal studies, the perturbed disc develop obvious degeneration after 6 weeks (Kaigle et al., Spine 22(24): 2796-2806, 1997), before which the relatively intact nucleus is still present as the provoked disc shows Grade III to IV changes on the Pfirrmann scale at just 5 weeks post-injury (Yoon, et al., supra). Therefore, at 2-5 weeks after the stab intervention, the statin-encapsulated copolymer is injected into the candidate discs (Grade II-III classified by Pfirrmann's categorization).

To monitor disc degeneration, IVD height index (DHI) has been utilized to report the physical severity of progressive disc degeneration (Lu et al., Spine 22(16): 1828-1834, 1997). Typically, DHI at different degeneration stage is compared to those of healthy disc at the predetermined levels, and the follow-up measurements of DHI can monitor if the condition of degenerative discs declines. It is expected that the DHI of the simvastatin treated group is approximately equal to or greater than 20% that of the control group (e.g., DHI is 90% of baseline for the treated group and 70% of baseline for the negative control group) with the standard deviation of 5% for both groups, and the MRI downgraded at least one to two degrees significantly to achieve comparable effectiveness of OP-1. Changes in the DHI of injected discs is expressed as percent DHT and normalized to the measured preoperative IVD height (percent DHI=postoperative DHI/preoperative DHI×100). MRI examination is conducted to track the changes in the degree and area of signal intensity after the treatment of simvastatin.

Quantitative statistical analysis is conducted to investigate the difference between treated and untreated discs in the DHI recovery, the volume of reconstructed nucleus, and the wet weight of NP (physically), the inflammatory responses and the phenotypical expressions described previously for the rat model and below (biochemically), and the degree of range of motion and the stiffness (biomechanically) using a the two-sample t-test or a Wilcoxon rank sum test.

The sample groups are divided into a combination of doses, for example, 4 doses of 0.05 mg/ml, 0.5 mg/ml, 5 mg/ml and 50 mg/ml, and multiple durations, for example 2, 4, 8, 12 and 24 weeks, in order to detect a minimally effective and statistically difference between two or more groups. To detect the difference between groups a two-sided, two-sample t-test at 0.0025 significance level is used, the doses and durations are adjusted as necessary.

In order to assess drug-induced intradical tissue repair, DNA content, as well as sGAG and type II collagen, as the key molecules that indicate the chondrogenic phenotype expression, are measured. It is anticipated that the delivered statin is effective if the increases in DNA, Collagen II, and sGAG reach statistical significance compared to untreated controls. A cell morphology and cloning score is also utilized. All of the nonparametric data (including the MRI and histological grading) is analyzed by Mann-Whiteny U test to judge significant differences. In one aspect, treatment with a hydrogel comprising a BMP activating agent, such as a statin, is considered effective for the degenerated disc if the positive responses within the same grade are significantly higher in the treated group than in the untreated one.

In one aspect, between 2 to 5 weeks post-injury is an appropriate time window to inject simvastatin-loaded polymer and a 2-5 week recovery time window to identify the candidates for the indication of the proposed treatment (Grade II-III) to give the injection.

After sacrifice, NP tissue samples from each group are dissected and saved for later RNA extraction. BMP-2, aggrecan, collagen type II and collagen type I gene expression is measured using real-time PCR. 3-5 NP tissue samples from each group are digested with papain at 60° C. for 48 hrs. The DNA content in the digest is analyzed by a fluorometric DNA assay using the bisbenzimidazole fluorescent (Hoechst 33258) method. BMP-2 protein in the digest is analyzed using a commercial ELISA kit. The papain digests are also used for measuring the content of sGAG by the dimethyl-methylene bule (DMMB) assay and the contents of hydroxyproline, as well measurement of collagen, using reverse-phase high-performance liquid chromatography after hydrolysis with 6-M hydrochloric acid for 16 hours at 120° C. and derivatization by phenylisothiocyanate. All biochemical data is normalized by wet weight.

For biomechanical testing, the spine segments containing untreated injured, treated, and intact discs from euthanized Yucatan minipigs are harvested and collected en bloc. All vertebral bodies of each vertebra-disc-vertebra segment are embedded in fixation cups using polymethylmethacrylate to facilitate the mechanical testing. The specimen is then solid engaged with the testing clamp and the well aligned such that the transverse plane of the disc remains horizontal and normal to the vertical loading axis. Saline-soaked gauze is wrapped around the discs during processing and testing to minimize the dehydration. Each specimen is mounted using the fixation cups into a hydraulic materials testing system that generates loading modes of extension, flexion, torsion, left lateral and right lateral bending under compression. Markers are placed at reference points of vertebral bodies and the coordinates of the marker points are recorded using a digitizer and converted into 3-D angular displacement to calculate the ranges of motion (ROM). Stiffness is also calculated based on the load-displacement curves.

Flexion-extension moments are applied to the specimen at the upper level using a specially designed testing machine and segmental spinal kinematics collected using an Optotrack (optoelectronic system). The range of motion (maximum flexion angle), maximum flexion moment, laxity zone (flexion and extension), and two stiffness properties are determined. The laxity zone is defined as the displacement corresponding to 1.0 Nm from the neutral position (Tencer et al., Spine 20:2408-2414, 1995). This is similar to the neutral zone measure used to reflect instability. Stiffness is quantified similarly to Wilke et al. (J. Orthop. Res. 14:500-3, 1996 and Wilke et al., Eur Spine J 7:148-54, 1998). The stiffness 1 (S1) is defined as the slope in the low-stiffness and stiffness 2 (S2) is defined as the slope in the high-stiffness region at the maximum load.

Tissue specimens are excised from the vertebral body-disc-vertebral body unit, fixed in 10% formalin, decalcified, embedded in paraffin and sectioned. Hematoxylin and eosin (H&E) staining is performed to evaluate the disc degeneration grade. The assessment of sulfated glycosaminoglycan (sGAG) content is performed after staining of the tissue sections with Safranin O/Fast Green. Immunohistochemical staining is performed to examine changes in collagen type I and type II content and distribution. Cellularity of nucleus pulposus is measured by H&E staining and TUNEL assay to evaluate potential negative effects of simvastatin treatment.

The IVD degeneration is graded along the time span based on a grading system described above and compared with the evaluation standard by histological morphology described by Thompson (Thompson et al., Spine 15(5): 411-415, 1990). The content of glycosaminoglycan (GAG) and the distribution of BMPRII receptor is also measured to observe the changes along the degeneration. Additionally, studies have shown that the expression of inflammatory cytokines that induce DDD symptoms were significantly altered with a decrease in IL-1 and an increase in IL-8 when a percutaneous plasma discectomy was applied. As such, the change in IL-1, IL-8 and other cytokine levels (e.g., TNF-α, IL-1β, and IL-6) between the treated and the untreated groups are analyzed at various timepoints using techniques standard in the art.

### Example 5

### Treatment of Disc or Joint Degeneration in Humans

The animal models described above provide guidance for administration of a controlled release formulation, such as a temperature sensitive hydrogel, comprising a BMP-2 activating agent, such as a statin, to human patients suffering from disc degeneration or other cartilage injury or cartilage related disorders.

It is contemplated that human patients are administered the composition described herein by injection of the controlled release formulation directly to the site of cartilage injury. In one aspect, the injection is carried out using a syringe of appropriate size and is aided by use of a fluoroscope to guide the syringe to the appropriate location of cartilage injury.

For example, in a patient suffering from disc degeneration, the injection treatment is similar to the procedures of discogram and percutaneous discectomy on human patients performed in a minimally invasive manner. Briefly, a radiolucent marker is placed on the skin to identify the optimal point of entry for percutaneous access to the disc. This entry point is typically 8-9 cm off of midline along the right-sided paramedial line. A 22 gauge spinal needle is fluoroscopically guided into the disc from a right posterolateral, extrapedicular approach entering the disc through an access triangle delineated medio-dorsally by the superior articular process, supero-ventrally by the exiting nerve root and caudally by the body of the superior endplate of the inferior vertebra. A small amount of radio-opaque tracer is injected through the needle and into the disc to assure correct placement and integrity of the disc. Composition-loaded polymer is then injected through the needle in those cartilage spaces deemed continent. The needle is removed and the puncture site is dressed in a sterile fashion.

Numerous studies in the art have been published providing additional guidance on methods to implant material into the spine or joints of patients. For example, Masshman et al (Spine 32(18):2027-2030, 2001) teach an image-guidance system for guidance on surgical procedures in the spine, compared to conventional fluoroscopy methods. Additionally, Mistry et al., (J Spinal Disord Tech 19(4):231-36, 2006) describe methods to accurately locate the midline and disc orientation in an individual and Wolf et al. (Spine 26(22):2472-77, 2001) describe methods to determine disc distance and tool orientation for spinal procedures for accurate implantation. These techniques are also useful to provide guidance for injection of the controlled release composition described herein.

Repair or improvement (including lack of progression of degeneration) in an individual receiving a controlled release formulation as described herein is measured using techniques known in the art. For example, MRI and CT scanning are used to assess the injured cartilage. Additional analytical tools include range of motion tests are also performed, including the and Oswestry Disability Index (McAfee et al., Neurosurg Focus 22(1): E13, 2007), a visual analog scale (VAS), heterotropic ossification analysis as measured by the McAffee scale (Heideke et al., Acta Neurochir 150(5):453-59, 2008), and personal questionnaire (e.g., Zurich Claudication Questionnaire, McAfee et al., Neurosurg Focus 22(1): E13, 2007)).

It is expected that injection of a controlled release formulation described herein into a site of cartilage injury improves the injured cartilage, such that the damage diminishes, or at least does not progress. Additionally, patients have improved range of motion and flexibility in the joint or disc with previous cartilage damage after treatment with the controlled release composition.

Numerous modifications and variations of the invention as set forth in the above illustrative examples are expected to occur to those skilled in the art. Consequently only such limitations as appear in the appended claims should be placed on the invention.

### REFERENCES

1. Adams M A, Roughley P J (2006) Spine 31:2151-2161.

2. Masuda K, An H S (2006) Eur Spine J 15 Suppl 3:S422-432.

3. Maniadakis N, Gray A (2000) Pain 84:95-103.

4. Luoma K, Riihimaki H, Luukkonen R, Raininko R, Viikari-Juntura E, Lamminen A (2000) Spine 25:487-492.

5. Chujo T, An H S, Akeda K, Miyamoto K, Muehleman C, Attawia M, Andersson G, Masuda K (2006) Spine 31:2909-2917.

6. Osada R, Ohshima H, Ishihara H, Yudoh K, Sakai K, Matsui H, Tsuji H (1996) J Orthop Res 14:690-699.

7. Li J, Yoon S T, Hutton W C (2004) J Spinal Disord Tech 17: 423-428.

8. Gruber H E, Fisher E C, Jr., Desai B, Stasky A A, Hoelscher G, Hanley E N, Jr. (1997) Exp Cell Res 235:13-21.

9. Thompson J P, Oegema T R, Jr., Bradford D S (1991) Spine 16:253-260.

10. Tim Yoon S, Su Kim K, Li J, Soo Park J, Akamaru T, Elmer W A, Hutton W C (2003) Spine 28:1773-1780.

11. Takegami K, An H S, Kumano F, Chiba K, Thonar E J, Singh K, Masuda K (2005) Spine J 5:231-238.

12. Zhang Y, An H S, Song S, Toofanfard M, Masuda K, Andersson G B, Thonar E J (2004) Am J Phys Med Rehabil 83:515-521.

13. Le Maitre C L, Richardson S M, Baird P, Freemont A J, Hoyland J A (2005) J Pathol 207:445-452.

14. Kim D J, Moon S H, Kim H, Kwon U H, Park M S, Han K J, Hahn S B, Lee H M (2003) Spine 28:2679-2684.

15. Mundy G, Garrett R, Harris S, Chan J, Chen D, Rossini G, Boyce B, Zhao M, Gutierrez G (1999) Science 286:1946-1949.

16. Emmanuele L, Ortmann J, Doerflinger T, Traupe T, Barton M (2003) Biochem Biophys Res Commun 302:67-72.

17. Song C, Guo Z, Ma Q, Chen Z, Liu Z, Jia H, Dang G (2003) Biochem Biophys Res Commun 308:458-462.

18. Maeda T, Matsunuma A, Kawane T, Horiuchi N (2001) Biochem Biophys Res Commun 280:874-877.

19. Hatano H, Maruo A, Bolander M E, Sarkar G (2003) J Orthop Sci 8:842-848.

20. Hunter C J, Matyas J R, Duncan N A (2004) J Anat 205:357-362.

21. Ichimura K, Tsuji H, Matsui H, Makiyama N (1991) J Spinal Disord 4:428-436.

22. Maldonado B A, Oegema T R, Jr. (1992) J Orthop Res 10:677-690.

23. Chelberg M K, Banks G M, Geiger D F, Oegema T R, Jr. (1995) J Anat 186 (Pt 1):43-53.

24. Hauselmann H J, Fernandes R J, Mok S S, Schmid T M, Block J A, Aydelotte M B, Kuettner K E, Thonar E J (1994) J Cell Sci 107 (Pt 1):17-27.

25. Horner H A, Roberts S, Bielby R C, Menage J, Evans H, Urban J P (2002) Spine 27:1018-1028.

26. Zimmerman L B, De Jesus-Escobar J M, Harland R M (1996) Cell 86:599-606.

27. Groppe J, Greenwald J, Wiater E, Rodriguez-Leon J, Economides A N, Kwiatkowski W, Affolter M, Vale W W, Belmonte J C, Choe S (2002) Nature 420:636-642.

28. Groppe J, Greenwald J, Wiater E, Rodriguez-Leon J, Economides A N, Kwiatkowski W, Baban K, Affolter M, Vale W W, Belmonte J C, et al. (2003) J Bone Joint Surg Am 85-A Suppl 3:52-58.

29. Kasai M, Satoh K, Akiyama T (2005) Genes Cells 10:777-783.

30. Gori F, Demay M B (2005) Exp Cell Res 304:287-292.

31. Li T F, Darowish M, Zuscik M J, Chen D, Schwarz E M, Rosier R N, Drissi H, O'Keefe R J (2006) J Bone Miner Res 21:4-16.

32. Kuo P L, Hsu Y L, Chang C H, Chang J K (2005) J Pharmacol Exp Ther 314:1290-1299.

33. Hallahan A R, Pritchard J I, Chandraratna R A, Ellenbogen R G, Geyer J R, Overland R P, Strand A D, Tapscott S J, Olson J M (2003) Nat Med 9:1033-1038.

34. Ruppert R, Hoffmann E, Sebald W (1996) Eur J Biochem 237:295-302.

35. Grunder T, Gaissmaier C, Fritz J, Stoop R, Hortschansky P, Mollenhauer J, Aicher W K (2004) Osteoarthritis Cartilage 12:559-567.

36. Yanagita M (2005) Cytokine Growth Factor Rev 16:309-317.

37. Surani M A, Kimber S J, Osborn J C (1983) J Embryol Exp Morphol 75:205-223.

38. Goldstein J L, Brown M S (1990) Nature 343:425-430.

39. Liao J K (2005) Am J Cardiol 96:24F-33F.

40. Dirks A J, Jones K M (2006) Am J Physiol Cell Physiol 291:C1208-1212.

41. Wong W W, Dimitroulakos J, Minden M D, Penn L Z (2002) Leukemia 16:508-519.

42. Aznar S, Lacal J C (2001) Cancer Lett 165:1-10.

43. Coleman M L, Marshall C J, Olson M F (2004) Nat Rev Mol Cell Biol 5:355-366.

44. Shaw R J, Cantley L C (2006) Nature 441:424-430.

45. Chandrasekhar S, Esterman M A, Hoffman H A (1987) Anal Biochem 161:103-108.

46. Dey P, Saphos C A, McDonnell J, Moore V L (1992) Connect Tissue Res 28:317-324.

47. Khattak S F, Spatara M, Roberts L, Roberts S C (2006) Biotechnol Lett 28:1361-1370.

48. Masuda K, Takegami K, An H, Kumano F, Chiba K, Andersson G B, Schmid T, Thonar E (2003) J Orthop Res 21:922-930.

1. A method to repair or retard cartilage damage comprising the step of administering to a subject in need thereof a composition that increases bone morphogenic protein (BMP) expression directly into a site of cartilage injury in a controlled release formulation, wherein said composition is released in said cartilage injury at a rate and an amount effective to permit repair of or retard said damage.

2. A method to repair or retard cartilage damage comprising the step of administering to a subject in need thereof a statin that increases bone morphogenic protein expression to a site of cartilage injury in a controlled release formulation, wherein said statin is released in said cartilage injury at a rate and an amount effective to permit repair of or retard said damage.

3. The method of claim 1, wherein the cartilage is in a spinal disc.

4. The method of claim 1, wherein the cartilage is in a joint.

5. The method of claim 4, wherein the joint is selected from the group consisting of a fibrous joint, a cartilaginous joint and a synovial joint.

6-7. (canceled)

8. The method of claim 1, wherein the administering is by injection.

9. The method of claim 8, wherein the injecting is carried out using a fluoroscope to guide a syringe carrying the controlled release formulation.

10. The method of claim 9, wherein the syringe is maintained at 4° C. and the controlled release formulation is in a fluid state.

11. The method of claim 1, wherein the cartilage injury shows an initial indication of damage and said method repairs said initial damage or retards additional damage.

12. The method of claim 1, wherein the BMP is selected from the group consisting of BMP-2 and BMP-7.

13. The method of claim 1, wherein the composition is selected from the group consisting of a statin or derivative thereof, an isoprenyl transferase inhibitor, BMP-2 and a BMP-activating agent.

14-17. (canceled)

18. The method of claim 1, wherein the controlled release formulation is a hydrogel.

19-20. (canceled)

21. The method of claim 18, wherein the hydrogel comprises a hydrophobic polymer and a hydrophilic polymer.

22-23. (canceled)

24. The method of claim 21, wherein the polymers are homopolymers or copolymers.

25-26. (canceled)

27. The method of claim 1, wherein administering the controlled release composition promotes proliferation of chondrocytes or chondrocyte-like cells in the damaged cartilage site.

28. (canceled)

29. The method of claim 1, wherein the subject is a mammal.

30-31. (canceled)

32. A controlled release formulation for administering directly into damaged cartilage comprising a composition that increases bone morphogenic protein (BMP) expression, wherein administration of the controlled release formulation into the cartilage provides the compound at a rate and an amount effective to permit repair of, to retard damage to or to prevent additional damage to said cartilage.

33. (canceled)

34. The controlled release formulation of claim 32, wherein the cartilage is in a spinal disc.

35. The controlled release formulation of claim 32, wherein the cartilage is in a joint.

36-38. (canceled)

39. The controlled release formulation of claim 32, wherein the BMP is selected from the group consisting of BMP-2 and BMP-7.

40-42. (canceled)

43. The controlled release formulation of claim 32, wherein the composition is selected from the group consisting of a statin or derivative thereof, an isoprenyl transferase inhibitor, BMP-2 and a BMP-activating agent.

44-47. (canceled)

48. The controlled release formulation of claim 32, wherein the controlled release formulation is a hydrogel.

49-50. (canceled)

51. The controlled release formulation of claim 48, wherein the hydrogel comprises a hydrophobic polymer and a hydrophilic polymer.

52-56. (canceled)

57. The controlled release formulation of claim 48, wherein the hydrophilic polymer in the hydrogel is in a range from about 10% to 50%, from about 20% to 40%, or from about 20% to 30%.

58. The controlled release formulation of claim 48, wherein the hydrophobic polymer in the hydrogel is in a range from 40% to 90%, from about 60% to 80%, or from about 70%-80%.

59. The controlled release formulation of any claim 48, wherein the composition in the hydrogel is in a range from 1 to 50 mg/ml.

60. (canceled)

61. A method for delivering a composition that increases bone morphogenic protein (BMP) expression directly into damaged cartilage in a controlled release formulation to a patient in need thereof comprising, administering the composition in the controlled release formulation through a syringe into a site of cartilage injury, wherein the syringe is guided to the site of injury using a fluoroscope, and
wherein administration of the controlled release formulation into the cartilage provides the composition at a rate and an amount effective to permit repair of, to retard damage to or to prevent additional damage to said cartilage.

62-63. (canceled)
